(19) United States
(12) Patent Application Publication
(10) Pub. No.: US 2025/0206808 A1
(43) Pub. Date: Jun. 26, 2025

(54) Antibodies Against Henipaviruses

(71) Applicant: Mapp Biopharmaceutical, Inc., San Diego, CA (US)

(72) Inventor: Brandyn WEST, San Diego, CA (US)

(21) Appl. No.: 18/991,145

(22) Filed: Dec. 20, 2024

Related U.S. Application Data
(60) Provisional application No. 63/614,371, filed on Dec. 22, 2023.

(51) Int. Cl. C07K 16/10; A61K 39/00; A61P 37/04

(52) U.S. Cl. CPC C07K 16/1027; A61P 37/04; A61K 2039/505; C07K 2317/24; C07K 2317/76; C07K 2317/94

(57) ABSTRACT

Provided herein are Henipavirus antibodies. These Henipavirus antibodies bind to the Henipavirus F glycoprotein and have neutralizing activity against different Henipavirus species and strains. These antibodies are used in methods of inducing an immune response and methods of inhibiting Henipavirus infection. Additionally provided are methods of treating an infectious disease using such antibodies.

### CROSS-REFERENCE TO RELATED APPLICATIONS

The present application claims the benefit of and priority to U.S. Provisional Application No. 63/614,371, filed Dec. 22, 2023, which is hereby incorporated by reference in its entirety.

### STATEMENT OF GOVERNMENT SUPPORT

This invention was made with government support under grant number U19 AI 142764 awarded by the National Institutes of Health. The government has certain rights in the invention.

### SEQUENCE LISTING

The instant application contains a Sequence Listing which has been submitted electronically in XML format and is hereby incorporated by reference in its entirety. The Sequence Listing was created on Dec. 20, 2024, is named “107942-1476629-seqlisting.xml” and is 18,085 bytes in size.

### FIELD OF INFECTIOUS DISEASE THERAPEUTICS

This application relates to compositions, methods, and kits of therapeutic antibodies for the treatment of Henipavirus infections.

### BACKGROUND

Nipah virus (NiV) is a highly pathogenic zoonotic virus that causes regular outbreaks of severe respiratory disease and encephalitis, and even death, in humans and animals in South and Southeast Asia. NiV is one of two viruses in the Henipavirus genus, with Hendra virus (HeV) being the other. Pteropus fruit bats serve as the natural reservoir for NiV, though pigs and other domestic animals are also susceptible to infection (Halpin, Hyatt et al., 2011, Singh, Dhama et al., 2019). NiV can be transmitted to humans directly from infected animals or through the consumption of contaminated foods, such as date palm sap containing saliva or urine from infected bats (Soman Pillai, Krishna et al., 2020). It can also spread from person-to-person through contact with respiratory secretions (Luby 2013). Human NiV cases range from asymptomatic to severe disease and death, with fatality rates estimated at 40-90% (Arunkumar, Chandni et al., 2019, Singh, Dhama et al., 2019). The first reported outbreak of NiV in humans occurred in 1998 in Malaysia, with more recent outbreaks in Bangladesh and India (Soman Pillai, Krishna et al., 2020). While human cases have so far been limited to Southeast Asia, evidence of Henipavirus infection has been detected in West African bats and pigs, raising concerns of additional spillover events (Hayman, Suu-Ire et al., 2008, Hayman, Wang et al., 2011). High population densities in areas where NiV is found and its ability to spread from person-to-person increase the risk that NiV spillover could develop into a global pandemic (Luby 2013). Currently, no licensed vaccines or treatments are available for human use against NiV or the closely related HeV. Thus, there is a need for an effective Henipavirus therapeutic for human use.

Nipah virus (NiV) was initially isolated and identified in 1999 during an outbreak of encephalitis and respiratory illness among people with close contact with pigs in Malaysia and Singapore. Its name originated from Sungai Nipah, a village in the Malaysian Peninsula. The Malaysian strain of Nipah virus is designated NiV_{M}. Flying foxes of the Pteropus genus were subsequently identified as the reservoir for NiV. CDC. Nipah Virus Distribution Map. Updated Feb. 11, 2021. Accessed Sep. 9, 2022, cdc.gov/vhf/nipah/outbreaks/distribution-map.html. In the 1999 outbreak, NiV caused relatively mild disease in pigs, but nearly 300 human cases with over 100 deaths were reported. More than one million pigs were euthanized to stop the outbreak, and since then, no subsequent cases (in either swine or human) have been reported in either Malaysia or Singapore. In 2001, NiV was again identified as the causative agent in an outbreak of human disease in Bangladesh. The Bangladesh strain of the Nipah virus is designated NiV_{B}. In the same year, another outbreak was identified retrospectively in Siliguri, India, with reports of person-to-person transmission in hospital settings (nosocomial transmission). CDC. Nipah Virus (NiV) Factsheet. Updated 2022. Accessed Jul. 15, 2022, cdc.gov/vhf/nipah/pdf/factsheet.pdf.

After exposure followed by an incubation period of 5-14 days, the NiV illness presents with 3-14 days of fever and headache, followed by drowsiness, disorientation, mental confusion, and in some cases, encephalitis (i.e., inflammation of the brain). These signs and symptoms can progress to coma within 24-48 hours of onset. Some patients experience a respiratory illness during the early part of the infection, and half of the patients showing severe neurological signs also showed pulmonary signs. CDC. Nipah Virus (NiV) Factsheet. Updated 2022. Accessed Jul. 15, 2022, cdc.gov/vhf/nipah/pdf/factsheet.pdf. Long-term sequelae following NiV infection have also been noted, including persistent convulsions and personality changes. Latent infections with subsequent reactivation of NiV and death have also been reported months and even years after exposure. Transmission of NiV to humans may occur after direct contact with infected bats, infected pigs, or other NiV-infected people. Person-to-person transmission is commonly seen in the family and caregivers of NiV-infected patients. Transmission also occurs because of direct exposure to infected bats. An example of transmission is the consumption of raw date palm sap contaminated with infectious bat excretions. Nipah Virus (NiV) Factsheet. Updated 2022. Accessed Jul. 15, 2022, cdc.gov/vhf/nipah/pdf/factsheet.pdf.

Unlike the large Malaysian NiV outbreak in 1999, small outbreaks occur almost annually in Bangladesh and have been reported several times in India. As of September 2023, 405 cases of NiV have been recorded in India and Bangladesh since the first cases of NiV were retrospectively identified in 2001. Since the initial outbreak, cases have spanned from a high of 79 documented cases in one year (in 2001) to single-digit cases recorded in 2009, 2015, 2017, and 2021. Except for 2002 and 2006, cases of NiV outbreaks have occurred each year in India and Bangladesh.

Like NiV, the Pteropus genus is the natural reservoir for Hendra virus (HeV). Although HeV infection in humans not common, HeV can also cause respiratory illness with severe flu-like signs and symptoms, which may progress to encephalitis. CDC. Hendra Virus Disease (HeV). In some cases, HeV infection can lead to death. See cdc.gov/vhf/hendra/pdf/factsheet.pdf.

HeV and NiV are two members of the Henipavirus genus within the Paramyxoviridae family. Paramyxovirus entry into host cells requires fusion of the viral membrane and the host plasma membrane. Dang, Ha V et al. “Broadly neutralizing antibody cocktails targeting Nipah virus and Hendra virus fusion glycoproteins.” Nature structural & molecular biology vol. 28, 5 (2021): 426-434. doi:10.1038/s41594-021-00584-8. This process is mediated by the interplay between two glycoproteins present at the viral surface: an attachment protein and a fusion protein. Id. These two glycoproteins are known as G and F, respectively, for NiV and HeV. G glycoprotein is a type II homotetrameric transmembrane protein with an ectodomain comprising a stalk and a C-terminal β-propeller head, and the latter domain is responsible for binding to ephrinB2 or ephrinB3 (cphrinB2/B3) receptors. Dang, Ha V et al. “An antibody against the F glycoprotein inhibits Nipah and Hendra virus infections.” Nature structural & molecular biology vol. 26, 10 (2019): 980-987. doi:10.1038/s41594-019-0308-9. F glycoprotein is a homotrimeric type I transmembrane protein that is synthesized as a premature F₀ precursor and cleaved by cathepsin L during endocytic recycling to yield the mature, disulfide-linked, F1 and F2 subunits. Id. Viral fusion proteins are believed to exist in a kinetically trapped metastable conformation at the virus surface. Id. Upon binding to ephrinB2/B3, NiV G has been proposed to undergo conformational changes leading to F triggering and insertion of the F hydrophobic fusion peptide into the target membrane. Id. Subsequent refolding into the more stable post-fusion F conformation drives merger of the viral and host membranes to form a pore for genome delivery to the cell cytoplasm, as shown for other paramyxoviruses and pneumoviruses. Id. Because the G and F glycoproteins are on the surface of the virion, they are candidate targets for virus-neutralizing antibodies.

mAbs offer a highly specific, potent, and generally safe antiviral drug platform, especially for nonhuman antigen targets. An experimental human mAb directed against NiV G glycoprotein, m102.4, has been tested in a Phase 1 safety trial. Playford, Elliott Geoffrey et al., “Safety, tolerability, pharmacokinetics, and immunogenicity of a human monoclonal antibody targeting the G glycoprotein of Henipaviruses in healthy adults: a first-in-human, randomized, controlled, phase 1 study.” The Lancet. Infectious diseases vol. 20, 4 (2020): 445-454. doi:10.1016/S1473-3099(19)30634-6. m102.4 has been administered under compassionate use guidelines for post-exposure prophylaxis of NiV in one individual in the U.S. and for HeV in 18 people in Australia (Playford, Munro et al., 2020). m102.4 is an anti-G glycoprotein human mAb isolated from a naïve phage display library (Zhu, Bossart et al., 2008) shown to potently neutralize NiV and HeV in vitro.

Tested in AGMs (Chlorocebus aethiops), m102.4 protected against the Malaysia strain of NiV (NiV_{M}) when given 5 and 7 days after challenge via the combined intratracheal and intranasal route (Geisbert, Mire et al., 2014). However, the same dose regimen failed to protect AGMs infected with the Bangladesh strain of NiV (NiV_{B}), but did protect when given on days 3 and 5 after exposure (Mire, Satterfield et al., 2016). While m102.4 has similar neutralization activity in vitro against both the NiV_{B} and NiV_{M} strains (Dong, Cross et al., 2020), NiV_{B} appears to be more virulent than NiV_{M} in AGMs. Aside from m102.4, there are currently no licensed vaccines or therapies that exist for patients infected with NiV. Henipavirus infection causes severe respiratory illness and encephalitis and can be deadly. As of October 2023, there are no FDA-approved vaccines or therapeutics for human use as protection against Henipavirus.

### SUMMARY

In some embodiments, provided herein is an isolated humanized antibody that binds to Henipavirus F glycoprotein, wherein the isolated humanized antibody comprises: (i) a heavy chain variable region (V_{H}) with an amino acid substitution mutation at position 25 relative to a sequence of SEQ ID NO: 9, optionally wherein the V_{H} amino acid substitution mutation is T25S relative SEQ ID NO: 9; and/or (ii) a light chain variable region (V_{L}) with an amino acid substitution mutation at position 24 relative to a sequence of SEQ ID NO: 10, optionally wherein the V_{L} amino acid substitution mutation is S24R relative to SEQ ID NO: 10.

In some embodiments, the isolated humanized antibody binds to an epitope located on a pre-fusion form of the Henipavirus F glycoprotein.

In some embodiments, the Henipavirus F glycoprotein is a Nipah virus (NiV) F glycoprotein or a Hendra virus (HeV) F glycoprotein.

In some embodiments, the isolated antibody can treat Nipah virus (NiV), Bangladesh strain of NiV (NiV_{B}), Malaysia strain of NiV (NiV_{M}), Cedar henipavirus (CedV), Ghanaian bat henipavirus (Kumasi virus; KV), Langya henipavirus (LayV), Mojiang henipavirus (MojV), and Hendra virus (HeV).

In some embodiments, the isolated humanized antibody comprises: a heavy chain complementarity-determining region (HCDR) comprising: (a) an HCDR1 comprising a sequence of SEQ ID NO: 1 (SGYSITSDYYW), or a variant HCDR1 in which 1, 2, 3, 4, or 5 amino acids are substituted relative to SEQ ID NO: 1 (SGYSITSDYYW); (b) an HCDR2 comprising a sequence of SEQ ID NO: 2 (VTYDGSN), or a variant HCDR2 in which 1, 2, 3, 4, or 5 amino acids are substituted relative to SEQ ID NO: 2 (VTYDGSN); and (c) an HCDR3 comprising a sequence of SEQ ID NO: 3 (RFGSSYWAMDYW), or a variant HCDR3 in which 1, 2, 3, 4, or 5 amino acids are substituted relative to SEQ ID NO: 3 (RFGSSYWAMDYW); and a light chain complementarity-determining region (LCDR) comprising: (a) an LCDR1 comprising a sequence of SEQ ID NO: 4 (RASSSVSYMH), or a variant LCDR1 in which 1, 2, 3, 4, or 5 amino acids are substituted relative to SEQ ID NO: 4 (RASSSVSYMH); (b) an LCDR2 comprising a sequence of SEQ ID NO: 5 (STSNLAS), or a variant LCDR2 in which 1, 2, 3, 4, or 5 amino acids are substituted relative to SEQ ID NO: 5 (STSNLAS); and (c) an LCDR3 comprising a sequence of SEQ ID NO: 6 (HQWYSYPWT), or a variant LCDR3 in which 1, 2, 3, 4, or 5 amino acids are substituted relative to SEQ ID NO: 6 (HQWYSYPWT).

In some embodiments, the isolated humanized antibody comprises: (i) an HCDR1 comprising a sequence of SEQ ID NO: 1 (SGYSITSDYYW); (ii) an HCDR2 comprising a sequence of SEQ ID NO: 2 (VTYDGSN); (iii) an HCDR3 comprising a sequence of SEQ ID NO: 3 (RFGSSYWAMDYW); (iv) an LCDR1 comprising a sequence of SEQ ID NO: 4 (RASSSVSYMH); (v) an LCDR2 comprising a sequence of SEQ ID NO: 5 (STSNLAS); and (vi) an LCDR3 comprising a sequence of SEQ ID NO: 6 (HQWYSYPWT).

In some embodiments, the V_{H} comprises an amino acid sequence having at least 95% identity to the sequence of SEQ ID NO: 7, 9, or 15, and the V_{L} sequence comprises an amino sequence having at 95% identity to the sequence of SEQ ID NO: 8 or 10.

In some embodiments, the V_{H} comprises at least 95% identity to the sequence of SEQ ID NO: 7, 9, or 15; and (a) an HCDR1 comprising the sequence of SEQ ID NO: 1, or a variant HCDR1 in which 1, 2, 3, 4, or 5 amino acids are substituted relative to the sequence of SEQ ID NO: 1; (b) an HCDR2 comprising the sequence of SEQ ID NO: 2, or a variant HCDR2 in which 1, 2, 3, 4, or 5 amino acids are substituted relative to the sequence of SEQ ID NO: 2; and (c) an HCDR3 comprising the sequence of SEQ ID NO: 3, or a variant HCDR3 in which 1, 2, 3, 4, or 5 amino acids are substituted relative to the sequence of SEQ ID NO: 3; and the V_{L} comprises at least 95% identity to the sequence of SEQ ID NO: 8 or 10, and (a) an LCDR1 comprising the sequence of SEQ ID NO: 4, or a variant LCDR1 in which 1, 2, 3, 4, or 5 amino acids are substituted relative to the sequence of SEQ ID NO: 4; (b) an LCDR2 comprising the sequence of SEQ ID NO: 5, or a variant LCDR2 in which 1, 2, 3, 4, or 5 amino acids are substituted relative to the sequence of SEQ ID NO: 5; and (c) an LCDR3 comprising the sequence of SEQ ID NO: 6, or a variant LCDR3 in which 1, 2, 3, 4, or 5 amino acids are substituted relative to the sequence of SEQ ID NO: 6.

In some embodiments, at least 1 or 2 of the substitutions are conservative substitutions, at least 50% of the substitutions are conservative substitutions, or all of the substitutions are conservative substitutions.

In some embodiments, (i) the V_{H} sequence comprises the sequence of SEQ ID NO: 7 and (ii) the V_{L} sequence comprises the sequence of SEQ ID NO: 8.

In some embodiments, (i) the V_{H} sequence comprises the sequence of SEQ ID NO: 15 and (ii) the V_{L} sequence comprises the sequence of SEQ ID NO: 8.

In some embodiments, (i) the HC sequence comprises an amino acid sequence having at least 95% identity to the sequence of SEQ ID NO: 16, and (ii) the LC sequence comprises an amino acid sequence having at least 95% identity to the sequence of SEQ ID NO: 17.

In some embodiments, (i) the HC sequence comprises the sequence of SEQ ID NO: 16, and (ii) the LC sequence comprises the sequence of SEQ ID NO: 17.

In some embodiments, the isolated humanized antibody further comprises at least one Fc mutation, wherein the at least one Fc mutation confers increased half-life.

In some embodiments, the at least one Fc mutation is selected from the group consisting of M430L and N436A.

In some embodiments, a humanized antibody that binds to the same epitope as any isolated humanized antibody disclosed above.

In some embodiments, a humanized antibody that competes for binding with any isolated humanized antibody disclosed above.

In some embodiments, the antibody is a non-natural antibody.

Also provided herein is a recombinant nucleic acid encoding the antibody or humanized antibody disclosed herein.

Also provided herein is an expression vector comprising a polynucleotide encoding a V_{H} sequence and/or a V_{L} sequence of the antibody or humanized antibody disclosed herein.

Also provided herein is a host cell that comprises an expression vector disclosed above.

In some embodiments, provided herein is a method of producing a humanized antibody, the method comprising culturing a host cell disclosed herein under conditions in which one or more polynucleotides encoding a heavy chain and/or a light chain are expressed.

In some embodiments, provided herein is a polypeptide comprising (i) a V_{H} sequence of SEQ ID NO: 7 or having at least 70% amino acid sequence identity to a sequence of SEQ ID NO: 7 and/or (ii) a V_{L} sequence of SEQ ID NO: 8 having at least 70% amino acid sequence identity to a sequence of SEQ ID NO: 8.

In some embodiments, provided herein is a polypeptide comprising (i) a V_{H} sequence of SEQ ID NO: 15 or having at least 70%, at least 80%, at least 85%, at least 90%, at least 95%, at least 96%, at least 97%, at least 98%, at least 99%, or more amino acid sequence identity to a sequence of SEQ ID NO: 15 and/or (ii) a V_{L} sequence of SEQ ID NO: 8 having at least 70%, at least 80%, at least 85%, at least 90%, at least 95%, at least 96%, at least 97%, at least 98%, at least 99%, or more amino acid sequence identity to a sequence of SEQ ID NO: 8.

In some embodiments, provided herein is a polypeptide comprising (i) a HC sequence of SEQ ID NO: 16 or having at least 70%, at least 80%, at least 85%, at least 90%, at least 95%, at least 96%, at least 97%, at least 98%, at least 99%, or more amino acid sequence identity to a sequence of SEQ ID NO: 16 and/or (ii) an LC sequence of SEQ ID NO: 17 having at least 70%, at least 80%, at least 85%, at least 90%, at least 95%, at least 96%, at least 97%, at least 98%, at least 99%, or more amino acid sequence identity to a sequence of SEQ ID NO: 17.

In some embodiments, provided herein is a pharmaceutical composition comprising the humanized antibody disclosed herein and a pharmaceutically acceptable carrier.

In some embodiments, the pharmaceutical composition comprises two or more different humanized antibodies disclosed herein.

In some embodiments, provided herein is a method of inducing an immune response and/or treating in infectious disease, the method comprising administering an effective amount of a pharmaceutical composition of embodiment 21 or 22 to a subject in need thereof. In some embodiments, the infectious disease is caused by a Henipavirus. In some embodiments, the infectious disease is an infection caused by NiV, NiV_{B}, NiV_{M}, CedV, KV, LayV, MojV, and/or HeV.

In some embodiments, the administering step of the pharmaceutical composition occurs after the subject has been exposed to a Henipavirus that causes the infectious disease and before onset of symptoms of the infectious disease in the subject.

In some embodiments, the administering step of the pharmaceutical composition occurs before the subject is exposed to a Henipavirus that causes the infectious disease.

### BRIEF DESCRIPTION OF THE DRAWINGS

FIG. 1A-B show efficacy of anti-F protein monoclonal antibodies (mAbs) in hamsters. FIG. 1A: Survival. Syrian golden hamsters were inoculated with a target dose of 5×10⁶ PFU of the Bangladesh strain of Nipah virus (NiV_{B}) intranasally instilled. At 24 hours post challenge, 5 animals per group were dosed with 5 mg/kg 1F5 or 12B2 by intraperitoneal (i.p.) administration. A single cohort of hamsters was treated with combined administration of 2.5 mg/kg each of 1F5 and 12B2 by the same route. The control animal was treated with PBS. The “all controls” cohort includes all control animals to date that were inoculated with the same virus stock using the same protocol and personnel. FIG. 1B: Percent change in weight from baseline. Open symbols indicate individual animals that succumbed to Nipah virus (NiV) disease.

FIG. 2 shows survival of African green monkeys (AGMs) treated 5 days after exposure to NiV_{B}. Animals were challenged intranasally with a target dose of 40,000 PFU NiV_{B} using a mucosal atomization device (MAD). Treated animals received 25 mg/kg of either hu1F5 or m102.4 5 days post-exposure (dpe). The control animal was untreated. Improvement in survival in hu1F5-treated animals was significant compared to m102.4-treated animals (P<0.005 by log-rank test).

FIG. 3 shows clinical symptoms observed in NiV_{B}-exposed AGMs. Solid gray boxes (i.e., where all four quadrants of a box are filled with the same shade of light gray) indicate that no observations were obtained because the animal had previously succumbed to disease. Note that no symptoms were observed in any animals beyond Day 19; data beyond Day 20 are not shown to simplify the figure. Ext cont=external control (i.e., control animals from other studies in the same laboratory using the same viral stock and the same protocol including MAD challenge).

FIG. 4 shows respiration rate of AGMs after intranasal MAD exposure to NiV_{B}. Open symbols indicate animals that succumbed to disease. Closed symbols indicate survivors. Ext Cont=external control (i.e., control animals from other studies in the same laboratory using the same viral stock and the same protocol including MAD challenge).

FIG. 5A-B show viral load in AGM plasma. FIG. 5A: Plaque assay. FIG. 5B: qRT-PCR. Dashed horizontal lines indicate the limit of detection for the assay. Open symbols indicate animals that succumbed to disease. Closed symbols indicate survivors. ext cont=external control (i.e., control animals from other studies in the same laboratory using the same viral stock and the same protocol including MAD challenge).

FIG. 6 shows a table of viral load in tissues taken at necropsy as determined by qRT-PCR. Values shown are log 10 average total copies/mL. <LOD=below the limit of detection. Ec=external control; LN=lymph node; RU=right upper; RM=right medial; RL=right lower; LU=left upper; LM=left medial; LL=left lower; CSC=cervical spinal cord.

FIG. 7A-D show efficacy of 10 mg/kg of hu1F5 in AGMs intranasally exposed to NiV_{B} via MAD. FIG. 7A: Survival. Animals were challenged intranasally with a target dose of 40,000 PFU NiV_{B} using a MAD. Treated animals received 10 mg/kg of hu1F5 intravenously 5 dpe. The contemporaneous control animal (referred to as “external control 2” in FIG. 3-6) was untreated. Improvement in survival in hu1F5-treated animals was not significant (P=0.08 by log-rank test) compared to the contemporaneous control but was significant compared to the grouped controls (n=3; P=0.02). FIG. 7B: Respiration rate. FIG. 7C: Viral load in AGM plasma by plaque assay. Dashed horizontal lines indicate the limit of detection for the assay. FIG. 7D: Viral load in plasma by qRT-PCR. Dashed horizontal lines indicate the limit of detection for the assay. Open symbols indicate animals that succumbed to disease. For FIG. 7B-D: closed symbols indicate survivors.

FIG. 8A-D show sequences for an exemplary humanized Henipavirus antibody hu1F5. FIG. 8A shows the V_{H} sequence of hu1F5 (SEQ ID NO: 7). FIG. 8B shows the V_{L} sequence of hu1F5 (SEQ ID NO: 8). FIG. 8C (V_{H}) and 8D (V_{L}) show sequence changes in the humanization of an exemplary Henipavirus antibody. In humanizing the antibody sequences, IgG germline sequences that were closest to the mouse Henipavirus antibody V_{H} (SEQ ID NO: 9) and V_{L} (SEQ ID NO: 10) sequences were selected. Using structural guided resurfacing, select amino acids within the V_{H} and V_{L} were mutated (in bold text). In each of FIG. 8C-D, the Henipavirus antibody V_{H} (FIG. 8C) and V_{L} (FIG. 8D) sequences shown in the sequence alignments are the mouse sequence (first lines; SEQ ID NOS: 9 and 10), an initial humanized sequence (second lines; SEQ ID NOS: 11 and 12), a mutated sequence with conservative amino acid substitutions (third lines; SEQ ID NOS: 13 and 14), and clone h1F5.2 (fourth lines; final hu1F5 sequence; SEQ ID NOS: 7 and 8). h1F5.2 is also generally referred to as hu1F5 in this disclosure. Boxes indicate complementarity determining region sequences (CDRs).

FIG. 9A-B show sequences for an exemplary humanized Henipavirus antibody hu1F5. FIG. 9A shows the V_{H} sequence of hu1F5 (SEQ ID NO: 15). FIG. 9B shows the V_{L} sequence of hu1F5 (SEQ ID NO: 8).

### DETAILED DESCRIPTION

### 1. Introduction

This application relates to humanized monoclonal antibodies (mAbs) that bind to the conserved F glycoprotein of Henipavirus.

Disclosed herein are humanized mAbs that bind to pre-fusion F glycoprotein of Henipavirus. Also provided herein are methods of using these antibodies to induce an immune response and/or treating a Hernipavirus infectious disease in a patient. One or more epitopes recognized by these humanized mAbs are conserved between HeV and NiV, including the strains NiV_{B} and NiV_{M}.

### 2. Definitions

As used in herein, the singular forms “a”, “an,” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example, ±20%, ±10%, or ±5%, are within the intended meaning of the recited value.

The term “antibody” (interchangeably with the term “immunoglobulin” in this disclosure) and the related terms refer to an immunoglobulin or its fragment that binds to a particular spatial and polar organization of another molecule. The terms “immunoglobulin” and “antibody” are used interchangeably in this disclosure. Immunoglobulins include various classes and isotypes, such as IgA, IgD, IgE, IgG (IgG1, IgG2, IgG3, or IgG4), IgM, and the like. An antibody can be an isolated or recombinant antibody that comprises the necessary variable region sequences to specifically bind an antigenic epitope. Therefore, an “antibody” as used herein is any form of an antibody of any class or subclass or fragment thereof that exhibits the desired biological activity, e.g., binding a specific target antigen. Thus, it is used in the broadest sense and specifically covers a monoclonal antibody (mAb; including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity. An antibody can be monoclonal or recombinant and can be prepared by laboratory techniques, such as by preparing continuous hybrid cell lines and collecting the secreted protein or by cloning and expressing nucleotide sequences or their mutagenized versions coding at least for the amino acid sequences required for binding. Antibodies, referenced herein, may have sequences derived from non-human antibodies, human sequences, chimeric sequences, and wholly synthetic sequences. The term “antibody” encompasses natural (naturally occurring), artificially modified, and artificially generated antibody forms, such as humanized, human, single-chain, chimeric, synthetic, recombinant, hybrid, mutated, grafted, and in vitro generated antibodies and their fragments. The term “antibody” also includes composite forms, including but not limited to fusion proteins containing an immunoglobulin moiety. “Antibody” also refers to non-quaternary antibody structures (such as camelids and camelid derivatives) and antigen-binding fragments of antibodies, minibodies, bispecific antibodies, nanobodies (also referred to as VHH fragments), and diabodies. See Siontorou C G. 2013, “Nanobodies as novel agents for disease diagnosis and therapy,” Int J Nanomedicine 8:4215-4227. Antibody fragments may include Fab, Fv, F(ab′)2, Fab′, scFv, dsFv, ds-scFv, Fd, dAb, Fc, and the like. A natural antibody digested by papain yields three fragments: two Fab fragments and one Fc fragment. The Fc fragment is dimeric and contains two CH2 and two CH3 heavy chain domains. CH3 domains interact to form a homodimer. See Yang et al., 2018, “Engineering of Fc Fragments with Optimized Physicochemical Properties Implying Improvement of Clinical Potentials for Fc-Based Therapeutics” Frontiers in Immunology 8:1860. In addition, aggregates, polymers, and conjugates of immunoglobulins or their fragments can be used where appropriate.

As used herein, the term “backbone” as used in “IgG backbone” or “IgA backbone” refers to portions of an antibody (an IgG antibody or an IgA antibody) excluding the variable domains or variable regions of the same antibody.

The term “monoclonal antibody” (mAb) as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used by the present disclosure may be made by a variety of techniques, including but not limited to the hybridoma method, recombinant DNA methods, and phage-display methods, and methods utilizing transgenic animals containing all or part of the human immunoglobulin loci.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site and a residual “Fc” fragment, a designation reflecting the ability to crystallize readily. Pepsin treatment yields an F(ab′)2 fragment with two antigen combining sites and is still capable of cross-linking antigen.

As used herein, “V-region” refers to an antibody variable region domain comprising the segments of Framework (FR) 1 (FR1), complementarity determining region (CDR) 1 (CDR1), FR2, CDR2, FR3, CDR3, FR4. The heavy chain V-region, V_{H}, is a consequence of the rearrangement of a V-gene, a D-gene, and a J-gene, in what is termed V(D)J recombination during B-cell differentiation. The light chain V-region, V_{L}, is a consequence of the rearrangement of a V-gene and a J-gene. The variability of the V-region is not usually evenly distributed through the variable domains of antibodies. It is typically concentrated in three segments called CDRs or hypervariable regions both in the light chain, i.e., LCDR1, LCDR2, and LCDR3; and the heavy chain variable domains, i.e., HCDR1, HCDR2, and HCDR3. The more highly conserved portions of the variable domains are in the framework (FR), and those framework domains are FR1, FR2, FR3, and FR4.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, for example, a V_{H} CDR3 (HCDR3) is in the variable domain of the heavy chain of the antibody in which it is found, whereas a V_{L} CDR3 (LCDR3) is the CDR3 from the variable domain of the light chain of the antibody in which it is found.

The amino acid sequences of the CDRs and framework regions can be determined using various well-known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al., Antibody-antigen interactions: Contact analysis and binding site topography, J. Mol. Biol., 262 (5), 732-745 (1996); and Martin et al., Proc. Natl Acad. Sci. USA, 86, 9268-9272 (1989); Martin, et al., Methods Enzymol., 203, 121-153, (1991); Pedersen et al., Immunomethods, 1, 126, (1992); and Rees et al., In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)). Unless otherwise stated explicitly, the CDRs and framework regions are defined by Kabat.

An “Fc region” refers to the constant region of an antibody excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modified Fc regions, e.g., that are modified to modulate effector function or other properties such as pharmacokinetics, stability, or production properties of an antibody. Fc regions also include variants that do not exhibit alterations in biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal et al., Mol Immunol 30:105-108, 1993).

As used herein, “neutralizing antibody” (NAb) refers to an antibody, for example, a monoclonal antibody, capable of disrupting a formed viral particle or inhibiting formation of a viral particle or prevention of binding to or infection of mammalian cells by a viral particle. As used herein, neutralizing antibodies are capable of neutralizing epitopes of one or more virus (e.g., Henipavirus) species. As such, neutralizing antibodies can act to prevent or reduce the incidence of viral infection.

A “humanized antibody” is generally a human immunoglobulin (recipient antibody) in which residues from one or more CDRs are replaced by residues from one or more CDRs of a non-human antibody (donor antibody). Thus, a humanized antibody is a non-human, chimeric antibody. The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. (See Jones et al., 1986, Nature, 321:522-25; Riechmann et al., 1988, Nature, 332:323-29; and Presta, 1992, Curr. Op. Struct. Biol., 2:593-96).

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

As used herein, the term “epitope” means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein, such as an antibody, binds can be determined using known techniques for epitope determination such as, for example, testing for antigen binding protein binding to antigen variants with different point mutations.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hair pinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

A “recombinant nucleic acid” generally refers to a nucleic acid produced by the combining of genetic material from more than one origin. A recombinant nucleic acid may refer to an isolated” nucleic acid, i.e., a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. As used herein, a “recombinant nucleic acid encoding an antibody” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from one or more polypeptide sequences specifically disclosed herein in one or more substitutions, deletions, additions and/or insertions.

As used herein, “therapeutic agent” refers to an agent that when administered to a patient suffering from a disease, in a therapeutically effective dose, will cure, or at least partially arrest the symptoms of the disease and complications associated with the disease.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment.

As used herein, the term “immune response” refers to refers to both the humoral immune response and the cell-mediated immune response. The humoral immune response involves the stimulation of the production of antibodies by B lymphocytes that, for example, neutralize infectious agents, block infectious agents from entering cells, block replication of said infectious agents, and/or protect host cells from infection and destruction. The cell-mediated immune response refers to an immune response that is mediated by T-lymphocytes and/or other cells, such as macrophages, against an infectious agent, exhibited by a vertebrate (e.g., a human), that prevents or ameliorates infection or reduces at least one symptom thereof.

As used herein, the terms “effective amount,” “effective dose,” “therapeutically effective amount,” or “therapeutically effective dose” may be used interchangeably and refer to an amount of a pharmaceutical composition disclosed herein that, when administered to a subject, is effective to treat a disease or disorder such that the symptoms of the disease or disorder are ameliorated, or the likelihood of the disease or disorder developing or progressing is decreased. The effective amount may also provide a prophylactic benefit, e.g., preventing or delaying the onset (or reoccurrence) of injury, disease, pathology or condition, or reducing the likelihood of the onset (or reoccurrence) of an injury, disease, pathology, or condition, or their symptoms. An effective amount is not, however, a dosage so large as to cause adverse side effects, such as hyperviscosity syndromes, pulmonary edema, congestive heart failure, and the like. A suitable dose of a pharmaceutical composition as described herein can depend on a variety of factors including the intended use of the Henipavirus antibody, the particular pharmaceutical composition used, and whether it is used concomitantly with other therapeutic agents. For example, a different dose of a Henipavirus antibody may be required to treat a subject with a Henipavirus infection as compared to the dose of a Henipavirus antibody required for use as a prophylaxis in the same subject. Other factors affecting the dose administered to the subject include, e.g., the type or extent of the Henipavirus infection. Generally, an effective amount may vary with the subject's age, condition, and sex, as well as the extent of the disease in the subject and can be determined by one of skill in the art. For example, a child subject may require administration of a different dosage of a Henipavirus antibody than an adult subject. Other factors can include, e.g., other medical disorders concurrently or previously affecting the subject, the age and general health of the subject, the genetic disposition of the subject, diet, time of administration, the route of administration, and the size (e.g., body weight, body surface, or organ size), the rate of excretion, drug combination, and any other additional therapeutics that are administered to the subject. It should also be understood that a specific dosage and treatment regimen for any particular subject also depends upon the judgment of the treating medical practitioner (e.g., doctor or nurse). An effective amount is also one in which any toxic or detrimental effects of the composition are outweighed by the therapeutically beneficial effects. The dosage of the effective amount may be adjusted by the individual physician or veterinarian in the event of any complication.

The term “contemporaneous control” refers to a control performed within a specific experiment. The term “external control” refers to a control performed in a different experiment but is related in some aspect to the specific experiment being discussed. The term “grouped control” refers to external controls that are grouped for comparison with contemporaneous control(s) and/or use in the specific experiment being discussed. The term “all controls” includes contemporaneous controls and external controls.

### 3. Henipavirus Antibodies

The Henipavirus antibodies of the present disclosure are humanized monoclonal antibodies (mAbs) that bind to the F glycoprotein F₀ of Henipavirus. In HeV, the F₀ is Uniprot ID 089342. In NiV, the F₀ is Uniprot ID Q9IH63. Without humanization, the murine-derived 1F5 mAb may generate an anti-drug antibody response, thereby having a decreased utility as a drug.

Humanization of the Henipavirus antibodies of the present disclosure are performed in silico, e.g., using the Mapp Biopharmaceutical in silico humanization pipeline. For each mAb, mouse constant heavy and light chain domains are exchanged with human sequences while the mouse CDR domains stay the same. High-resolution homology models are constructed of each variable domain, and a structure-guided resurfacing approach is employed to maintain the murine CDR conformations and antigen binding while reducing deviations from the inferred human germline progenitor sequences. Initial humanization is followed by iterative rounds of structure-guided engineering to eliminate residues that contribute to chemical liability hotspots such as aggregation, glycosylation, and enzymatic cleavage.

Henipavirus infectious diseases include any disease developed from infection of Henipavirus. Nonlimiting examples of Henipaviruses include NiV, NiV_{B}, NiV_{M}, Cedar henipavirus (CedV), Ghanaian bat henipavirus (Kumasi virus; KV), Langya henipavirus (LayV), Mojiang henipavirus (MojV), and HeV. See, Quarleri, Jorge et al., “Henipaviruses: an expanding global public health concern?.” GeroScience vol. 44, 5 (2022): 2447-2459. doi:10.1007/s11357-022-00670-9.

a. Structures of Henipavirus Antibodies
i. Complementarity Determining Regions (CDRs)

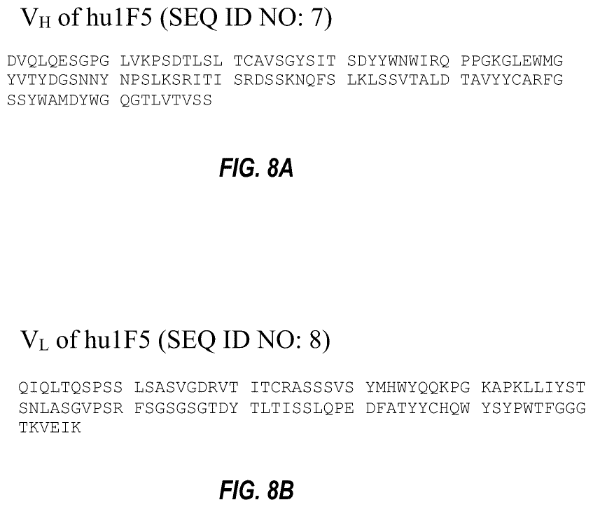

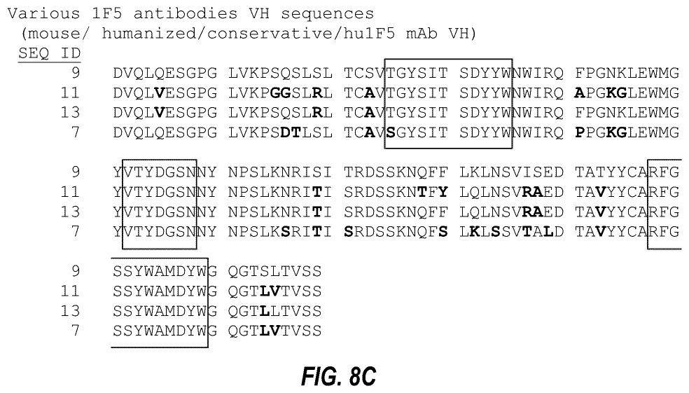

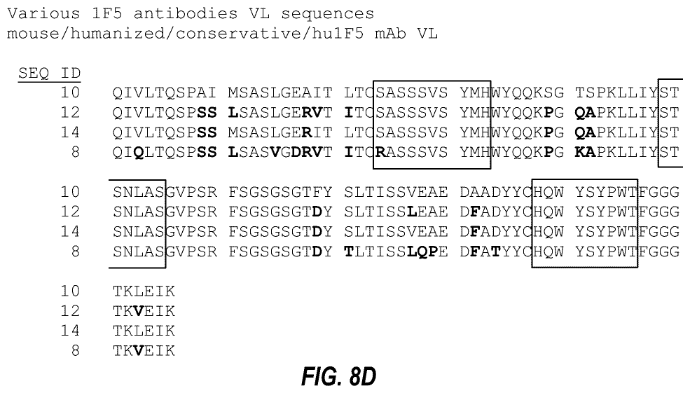

In some embodiments, the Henipavirus antibody includes certain CDR amino acids or mutations in its HCDR and/or LCDR sequences. In some embodiments, the Henipavirus antibody HCDR sequence comprises a threonine or a serine at the first amino acid of HCDR1. In some embodiments, the Henipavirus antibody HCDR sequence comprises a serine at the first amino acid of HCDR1. In some embodiments, the Henipavirus antibody HCDR sequence comprises a T25S mutation at the first amino acid of HCDR1. In some embodiments, the first amino acid of HCDR1 corresponds to the amino acid at position 1 of SEQ ID NO: 1 and/or the amino acid at position 25 of the antibody V_{H}. In some embodiments, the reference sequence for the antibody V_{H} is SEQ ID NO: 9 (FIG. 8C). In some embodiments, the Henipavirus antibody LCDR sequence comprises an arginine or a serine at the first amino acid of LCDR1. In some embodiments, the Henipavirus antibody LCDR sequence comprises an arginine at the first amino acid of LCDR1. In some embodiments, the Henipavirus antibody LCDR sequence comprises a S24R mutation at the first amino acid of LCDR1. In some embodiments, the first amino acid of LCDR1 corresponds to the amino acid at position 1 of SEQ ID NO: 4 and/or the amino acid at position 24 of the antibody V_{L}. In some embodiments, the reference sequence for the antibody V_{L} is SEQ ID NO: 10 (FIG. 8D).

In some embodiments, the Henipavirus antibody HCDR sequence comprises (a) a HCDR1 of SEQ ID NO: 1 or a HCDR1 variant of SEQ ID NO: 1 in which 1, 2, 3, 4, or 5 amino acids are substituted; (b) a HCDR2 of SEQ ID NO: 2 or a HCDR2 variant of SEQ ID NO: 2 in which 1, 2, 3, 4, or 5 amino acids are substituted; and (c) a HCDR3 of SEQ ID NO: 3 or a HCDR3 variant of SEQ ID NO: 3 in which 1, 2, 3, 4, or 5 amino acids are substituted. In some embodiments, the Henipavirus antibody LCDR sequence comprises (a) a LCDR1 of SEQ ID NO: 4 or a LCDR1 variant of SEQ ID NO: 4 in which 1, 2, 3, 4, or 5 amino acids are substituted; (b) a

LCDR2 of SEQ ID NO: 5 or a LCDR2 variant of SEQ ID NO: 5 in which 1, 2, 3, 4, or 5 amino acids are substituted; and (c) a LCDR3 of SEQ ID NO: 6 or a LCDR3 variant of SEQ ID NO: 6 in which 1, 2, 3, 4, or 5 amino acids are substituted.

In some embodiments, the Henipavirus antibody comprises (a) a HCDR sequence comprising a HCDR1 of SEQ ID NO: 1, a HCDR2 of SEQ ID NO: 2, and a HCDR3 of SEQ ID NO: 3; and (b) a LCDR sequence comprising a LCDR1 of SEQ ID NO: 4, a LCDR2 of SEQ ID NO: 5, and a LCDR3 of SEQ ID NO: 6.

ii. Heavy Chain and Light Chain Variable Regions (V_{H} and V_{L})

In some embodiments, the Henipavirus antibody V_{H} comprises an amino acid sequence having at least 10%, at least 20%, at least 30%, at least 40%, at least 50%, at least 60%, at least 70%, at least 80%, at least 85% at least 90%, at least 95%, at least 96%, at least 97%, at least 98%, at least 99%, or more identity to the sequence of SEQ ID NO: 7 (FIG. 8A).

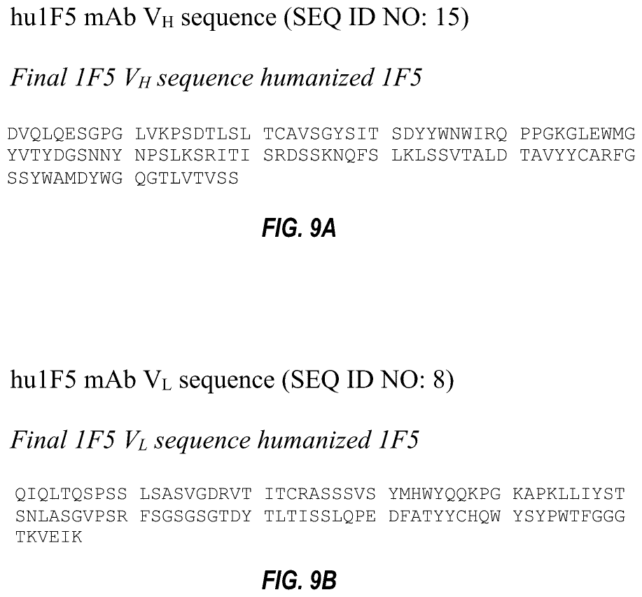

In some embodiments, the Henipavirus antibody V_{H} comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 15 (FIG. 9A).

In some embodiments, the Henipavirus antibody V_{L} comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 8 (FIG. 8B and FIG. 9B).

In some embodiments, the Henipavirus antibody V_{H} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 7 (FIG. 8A).

In some embodiments, the Henipavirus antibody V_{H} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 15 (FIG. 9A).

In some embodiments, the Henipavirus antibody V_{L} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 8 (FIG. 8B and FIG. 9B).

In some embodiments, the Henipavirus antibody heavy chain (HC) comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 16.

In some embodiments, the Henipavirus antibody light chain (LC) comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 17.

In some embodiments, the Henipavirus antibody V_{H} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 16. (MBP1F5 HC)

In some embodiments, the Henipavirus antibody V_{L} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 17. (Final MBP1F5 LC)

iii. Framework Regions

In some embodiments, the Henipavirus antibody includes certain framework (FR) amino acids or mutations in its V_{H} and/or V_{L}. In some embodiments, the FR mutations, when compared to their reference (e.g., murine) sequences, include amino acid substitutions, e.g., conservative amino acid substitutions. In some cases, mutations are introduced to the CDR region and/or framework regions of the reference sequences (e.g., the murine heavy chain sequence SEQ ID NO: 9, and murine light chain sequence SEQ ID NO: 10) improve useful properties of the antibody. For example, by identifying the closest germline sequence and using a structural guided approach, these mutations do not impair the antibody's binding affinity but can improve stability of the antibody structure. See, e.g., FIG. 8C-D.

In some embodiments, the Henipavirus antibody V_{H}, using SEQ ID NO: 9 as a reference V_{H} sequence, comprises an amino acid corresponding to position 25 of SEQ ID NO: 9 that is not threonine. In some embodiments, that amino acid corresponding to position 25 of SEQ ID NO: 9 is alanine, arginine, asparagine, aspartic acid, cysteine, glutamic acid, glutamine, glycine, histidine, isoleucine, leucine, lysine, methionine, phenylalanine, proline, serine, tryptophan, tyrosine, or valine. In some embodiments, that amino acid corresponding to position 25 of SEQ ID NO: 9 is serine.

In some embodiments, the Henipavirus antibody V_{L}, using SEQ ID NO: 10 as a reference V_{L} sequence, comprises an amino acid corresponding to position 24 of SEQ ID NO: 10 that is not serine. In some embodiments, that amino acid corresponding to position 24 of SEQ ID NO: 10 is alanine, arginine, asparagine, aspartic acid, cysteine, glutamic acid, glutamine, glycine, histidine, isoleucine, leucine, lysine, methionine, phenylalanine, proline, threonine, tryptophan, tyrosine, or valine. In some embodiments, that amino acid corresponding to position 24 of SEQ ID NO: 10 is arginine.

In some embodiments, the amino acid substitutions are conservative amino acid substitutions. In some embodiments, the FR region(s) have at least 1 or 2 conservative substitutions, or at least 50% conservative substitutions. In some embodiments, all the substitutions are conservative substitutions. In some embodiments, the Henipavirus antibody V_{H}, using SEQ ID NO: 9 as a reference V_{H} sequence, comprises one or more of the following amino acids: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115. In some embodiments, the Henipavirus antibody V_{H}, using SEQ ID NO: 9 as a reference V_{H} sequence, comprises all of the following amino acids: a glutamine at position 5; a serine at position 15; an aspartate at position 16; a threonine at position 17; an serine at position 19; an alanine at position 23; a serine at position 25; a proline at position 41; a lysine at position 44; a glycine at position 45; a serine at position 66; a threonine at position 69; a serine at position 71; a glutamine at position 78; a tyrosine or a serine at position 80; a lysine at position 82; a serine at position 84; a threonine at position 87; an alanine at position 88; a leucine at position 89; a valine at position 93; a leucine at position 114; and a valine at position 115.

In some embodiments, the Henipavirus antibody V_{L}, using SEQ ID NO: 10 as a reference V_{L} sequence, comprises one or more of the following amino acids: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or arginine at position 24; a proline at position 39; a glutamine or a lysine at position 41; an alanine at position 42; an aspartate at position 69; a serine or a threonine at position 71; a leucine or a valine at position 77; a glutamate or a glutamine at position 78; an alanine or a proline at position 79; a phenylalanine at position 82; an aspartate or a threonine at position 84; and a valine or a leucine at position 103. In some embodiments, the Henipavirus antibody V_{L}, using SEQ ID NO: 10 as a reference V_{L} sequence, comprises all of the following amino acids: a glutamine at position 3; a serine at position 9; an serine at position 10; a leucine at position 11; a valine at position 15; an aspartate at position 17; an arginine at position 18; a valine at position 19; a isoleucine at position 21; an arginine at position 24; a proline at position 39; a lysine at position 41; an alanine at position 42; an aspartate at position 69; a threonine at position 71; a leucine at position 77; a glutamine at position 78; a proline at position 79; a phenylalanine at position 82; a threonine at position 84; and a valine at position 103.

In some embodiments, the Henipavirus antibody comprises (a) a V_{H}, using SEQ ID NO: 9 as a reference V_{H} sequence, comprising all of the following amino acids: a glutamine at position 5; a serine at position 15; an aspartate at position 16; a threonine at position 17; an serine at position 19; an alanine at position 23; a serine at position 25; a proline at position 41; a lysine at position 44; a glycine at position 45; a serine at position 66; a threonine at position 69; a serine at position 71; a glutamine at position 78; a tyrosine or a serine at position 80; a lysine at position 82; a serine at position 84; a threonine at position 87; an alanine at position 88; a leucine at position 89; a valine at position 93; a leucine at position 114; and a valine at position 115; and (b) a V_{L}, using SEQ ID NO: 10 as a reference V_{L} sequence, all of the following amino acids: a glutamine at position 3; a serine at position 9; an serine at position 10; a leucine at position 11; a valine at position 15; an aspartate at position 17; an arginine at position 18; a valine at position 19; a isoleucine at position 21; an arginine at position 24; a proline at position 39; a lysine at position 41; an alanine at position 42; an aspartate at position 69; a threonine at position 71; a leucine at position 77; a glutamine at position 78; a proline at position 79; a phenylalanine at position 82; a threonine at position 84; and a valine at position 103.

iv. Combinations of CDR and Framework Region Variations

In some embodiments, the Henipavirus antibody comprises a V_{H} and a V_{L}, wherein the V_{H} comprises an amino acid sequence having at least 95% identity to the sequence of SEQ ID NO: 9 (FIG. 8C), and an amino acid corresponding to position 25 of SEQ ID NO: 9 that is any amino acid except threonine. In some embodiments, that amino acid corresponding to position 25 of SEQ ID NO: 9 is alanine, arginine, asparagine, aspartic acid, cysteine, glutamic acid, glutamine, glycine, histidine, isoleucine, leucine, lysine, methionine, phenylalanine, proline, serine, tryptophan, tyrosine, or valine. In some embodiments, that amino acid corresponding to position 25 of SEQ ID NO: 9 is serine.

In some embodiments, the Henipavirus antibody comprises a V_{H} and a V_{L}, wherein the V_{L} comprises an amino acid sequence having at least 95% identity to the sequence of SEQ ID NO: 10 (FIG. 8D), and an amino acid corresponding to position 24 of SEQ ID NO: 10 that is any amino acid except serine. In some embodiments, that amino acid corresponding to position 24 of SEQ ID NO: 10 is alanine, arginine, asparagine, aspartic acid, cysteine, glutamic acid, glutamine, glycine, histidine, isoleucine, leucine, lysine, methionine, phenylalanine, proline, threonine, tryptophan, tyrosine, or valine. In some embodiments, that amino acid corresponding to position 24 of SEQ ID NO: 10 is arginine.

In some embodiments, the Henipavirus antibody comprises a V_{H} and a V_{L}, wherein (a) the V_{H} comprises (i) a HCDR1 of SEQ ID NO: 1 or a HCDR1 variant of SEQ ID NO: 1 in which 1, 2, 3, 4, or 5 amino acids are substituted; (ii) a HCDR2 of SEQ ID NO: 2 or a HCDR2 variant of SEQ ID NO: 2 in which 1, 2, 3, 4, or 5 amino acids are substituted; (iii) a HCDR3 of SEQ ID NO: 3 or a HCDR3 variant of SEQ ID NO: 3 in which 1, 2, 3, 4, or 5 amino acids are substituted; and (iv) one or more of the following mutations when SEQ ID NO: 9 is the V_{H} reference sequence: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115; and (b) the V_{L} comprises (i) a LCDR1 of SEQ ID NO: 4 or a LCDR1 variant of SEQ ID NO: 4 in which 1, 2, 3, 4, or 5 amino acids are substituted; (ii) a LCDR2 of SEQ ID NO: 5 or a LCDR2 variant of SEQ ID NO: 5 in which 1, 2, 3, 4, or 5 amino acids are substituted; (iii) a LCDR3 of SEQ ID NO: 6 or a LCDR3 variant of SEQ ID NO: 6 in which 1, 2, 3, 4, or 5 amino acids are substituted; and (iv) one or more of the following mutations when SEQ ID NO: 10 is the V_{L} reference sequence: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or an arginine at position 24, a proline at position 39; a glutamine or a lysine at position 41; an alanine at position 42; an aspartate at position 69; a serine or a threonine at position 71; a leucine or a valine at position 77; a glutamate or a glutamine at position 78; an alanine or a proline at position 79; a phenylalanine at position 82; an aspartate or a threonine at position 84; and a valine or a leucine at position 103.

In some embodiments, the Henipavirus antibody V_{H} comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 9 (FIG. 8C), and one or more of the following mutations when SEQ ID NO: 9 is the Vu reference sequence: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115.

In some embodiments, the Henipavirus antibody V_{H} comprises an amino acid sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 9 (FIG. 8C), and all of the following mutations when SEQ ID NO: 9 is the V_{H} reference sequence: a glutamine at position 5; a serine at position 15; an aspartate at position 16; a threonine at position 17; an serine at position 19; an alanine at position 23; a serine at position 25; a proline at position 41; a lysine at position 44; a glycine at position 45; a serine at position 66; a threonine at position 69; a serine at position 71; a glutamine at position 78; a serine at position 80; a lysine at position 82; a serine at position 84; a threonine at position 87; an alanine at position 88; a leucine at position 89; a valine at position 93; a leucine at position 114; and a valine at position 115.

In some embodiments, the Henipavirus antibody V_{H} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 9 (FIG. 8C), and one or more of the following mutations when SEQ ID NO: 9 is the Vu reference sequence: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115.

In some embodiments, the Henipavirus antibody V_{H} comprises an amino acid sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 9 (FIG. 8C), and all of the following mutations when SEQ ID NO: 9 is the Vu reference sequence: a glutamine at position 5; a serine at position 15; an aspartate at position 16; a threonine at position 17; an serine at position 19; an alanine at position 23; a serine at position 25; a proline at position 41; a lysine at position 44; a glycine at position 45; a serine at position 66; a threonine at position 69; a serine at position 71; a glutamine at position 78; a serine at position 80; a lysine at position 82; a serine at position 84; a threonine at position 87; an alanine at position 88; a leucine at position 89; a valine at position 93; a leucine at position 114; and a valine at position 115.

In some embodiments, the Henipavirus antibody V_{L} comprises an amino sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 10 (FIG. 8D), and one or more of the following mutations when SEQ ID NO: 10 is the V_{L} reference sequence: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or an arginine at position 24, a proline at position 39; a glutamine or a lysine at position 41; an alanine at position 42; an aspartate at position 69; a serine or a threonine at position 71; a leucine or a valine at position 77; a glutamate or a glutamine at position 78; an alanine or a proline at position 79; a phenylalanine at position 82; an aspartate or a threonine at position 84; and a valine or a leucine at position 103.

In some embodiments, the Henipavirus antibody V_{L} comprises an amino sequence having at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 10 (FIG. 8D), and all of the following mutations when SEQ ID NO: 10 is the V_{L} reference sequence: a glutamine at position 3; a serine at position 9; an serine at position 10; a leucine at position 11; a valine at position 15; an aspartate at position 17; an arginine at position 18; a valine at position 19; a isoleucine at position 21; an arginine at position 24; a proline at position 39; a lysine at position 41; an alanine at position 42; an aspartate at position 69; a threonine at position 71; a leucine at position 77; a glutamine at position 78; a proline at position 79; a phenylalanine at position 82; a threonine at position 84; and a valine at position 103.

In some embodiments, and the Henipavirus antibody V_{L} comprises an amino sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 10 (FIG. 8D), and one or more of the following mutations when SEQ ID NO: 10 is the V_{L} reference sequence: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or an arginine at position 24; a proline at position 39; a glutamine or a lysine at position 41; an alanine at position 42; an aspartate at position 69; a serine or a threonine at position 71; a leucine or a valine at position 77; a glutamine or a glutamine at position 78; an alanine or a proline at position 79; a phenylalanine at position 82; an aspartate or a threonine at position 84; and a valine or a leucine at position 103.

In some embodiments, and the Henipavirus antibody V_{L} comprises an amino sequence having about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 10 (FIG. 8D), and all of the following mutations when SEQ ID NO: 10 is the V_{L} reference sequence: a glutamine at position 3; a serine at position 9; an serine at position 10; a leucine at position 11; a valine at position 15; an aspartate at position 17; an arginine at position 18; a valine at position 19; a isoleucine at position 21; an arginine at position 24; a proline at position 39; a lysine at position 41; an alanine at position 42; an aspartate at position 69; a threonine at position 71; a leucine at position 77; a glutamine at position 78; a proline at position 79; a phenylalanine at position 82; a threonine at position 84; and a valine at position 103.

In some embodiments, the Henipavirus antibody comprises a V_{H} and a V_{L} wherein (a) the V_{H} comprises (i) at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 9 (FIG. 8C); (ii) a HCDR1 of SEQ ID NO: 1 or a HCDR1 variant of SEQ ID NO: 1 in which 1, 2, 3, 4, or 5 amino acids are substituted; (iii) a HCDR2 of SEQ ID NO: 2 or a HCDR2 variant of SEQ ID NO: 2 in which 1, 2, 3, 4, or 5 amino acids are substituted; (iv) a HCDR3 of SEQ ID NO: 3 or a HCDR3 variant of SEQ ID NO: 3 in which 1, 2, 3, 4, or 5 amino acids are substituted; and (v) one or more of the following mutations when SEQ ID NO: 9 is the Vu reference sequence: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115; and (b) the V_{L} comprises (i) at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or more identity to the sequence of SEQ ID NO: 10 (FIG. 8D); and comprises (ii) a LCDR1 of SEQ ID NO: 4 or a LCDR1 variant of SEQ ID NO: 4 in which 1, 2, 3, 4, or 5 amino acids are substituted; (iii) a LCDR2 of SEQ ID NO: 5 or a LCDR2 variant of SEQ ID NO: 5 in which 1, 2, 3, 4, or 5 amino acids are substituted; (iv) a LCDR3 of SEQ ID NO: 6 or a LCDR3 variant of SEQ ID NO: 6 in which 1, 2, 3, 4, or 5 amino acids are substituted; and (v) one or more of the following mutations when SEQ ID NO: 10 is the V_{L} reference sequence: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or an arginine at position 24; a proline at position 39; a glutamine or a lysine at position 41; an alanine at position 42; an aspartate at position 69; a serine or a threonine at position 71; a leucine or a valine at position 77; a glutamate or a glutamine at position 78; an alanine or a proline at position 79; a phenylalanine at position 82; an aspartate or a threonine at position 84; and a valine or a leucine at position 103.

In some embodiments, the Henipavirus antibody comprises a V_{H} and a V_{L}, wherein (a) the V_{H} comprises (i) about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 9 (FIG. 8C); (ii) a HCDR1 of SEQ ID NO: 1 or a HCDR1 variant of SEQ ID NO: 1 in which 1, 2, 3, 4, or 5 amino acids are substituted; (iii) a HCDR2 of SEQ ID NO: 2 or a HCDR2 variant of SEQ ID NO: 2 in which 1, 2, 3, 4, or 5 amino acids are substituted; and (iv) a HCDR3 of SEQ ID NO: 3 or a HCDR3 variant of SEQ ID NO: 3 in which 1, 2, 3, 4, or 5 amino acids are substituted; and (v) one or more of the following mutations when SEQ ID NO: 9 is the V_{H} reference sequence: a valine or a glutamine at position 5; a glycine or a serine at position 15; a glutamine, a glycine, or an aspartate at position 16; a serine or a threonine at position 17; an arginine or a serine at position 19; an alanine at position 23; a threonine or a serine at position 25; an alanine, a phenylalanine, or a proline at position 41; an asparagine or a lysine at position 44; a lysine or a glycine at position 45; an asparagine or a serine at position 66; a threonine at position 69; a serine at position 71; a threonine or a glutamine at position 78; a tyrosine, a phenylalanine, or a serine at position 80; a glutamine or a lysine at position 82; an asparagine or a serine at position 84; an arginine or a threonine at position 87; an alanine at position 88; a glutamine or a leucine at position 89; a valine at position 93; a leucine at position 114; and a leucine or a valine at position 115; and (b) the V_{L} comprises (i) about 10% to about 99%, about 20% to about 90%, about 30% to about 80%, about 40% to about 70%, about 50% to about 95%, about 60% to about 85%, or about 70% to about 90%, or about 85% to about 95% identity to the sequence of SEQ ID NO: 10 (FIG. 8D); (ii) a LCDR1 of SEQ ID NO: 4 or a LCDR1 variant of SEQ ID NO: 4 in which 1, 2, 3, 4, or 5 amino acids are substituted; (iii) a LCDR2 of SEQ ID NO: 5 or a LCDR2 variant of SEQ ID NO: 5 in which 1, 2, 3, 4, or 5 amino acids are substituted; and (iv) a LCDR3 of SEQ ID NO: 6 or a LCDR3 variant of SEQ ID NO: 6 in which 1, 2, 3, 4, or 5 amino acids are substituted; and (v) one or more of the following mutations when SEQ ID NO: 10 is the V_{L} reference sequence: a valine or a glutamine at position 3; a serine at position 9; an serine at position 10; a leucine or a methionine at position 11; a leucine or a valine at position 15; a glutamate or an aspartate at position 17; an arginine at position 18; a valine or an isoleucine at position 19; a isoleucine or leucine at position 21; a serine or an arginine at position 24; a proline at position 39; a glutamine or a lysine at position 41; an alanine at position 42; an aspartate at position 69; a serine or a threonine at position 71; a leucine or a valine at position 77; a glutamate or a glutamine at position 78; an alanine or a proline at position 79; a phenylalanine at position 82; an aspartate or a threonine at position 84; and a valine or a leucine at position 103.

In some embodiments, the Henipavirus antibody comprises a V_{H} and a V_{L} wherein (a) the V_{H} comprises (i) a HCDR1 of SEQ ID NO: 1; (ii) a HCDR2 of SEQ ID NO: 2; (iii) a HCDR3 of SEQ ID NO: 3; and using SEQ ID NO: 9 as a reference V_{H} sequence, all of the following amino acids: a glutamine at position 5; a serine at position 15; an aspartate at position 16; a threonine at position 17; an serine at position 19; an alanine at position 23; a serine at position 25; a proline at position 41; a lysine at position 44; a glycine at position 45; a serine at position 66; a threonine at position 69; a serine at position 71; a glutamine at position 78; a serine at position 80; a lysine at position 82; a serine at position 84; a threonine at position 87; an alanine at position 88; a leucine at position 89; a valine at position 93; a leucine at position 114; and a valine at position 115; and (b) the V_{L} comprises (i) a LCDR1 of SEQ ID NO: 4; a LCDR2 of SEQ ID NO: 5; and a LCDR3 of SEQ ID NO: 6; and using SEQ ID NO: 10 as a reference V_{L} sequence, all of the following amino acids: a glutamine at position 3; a serine at position 9; an serine at position 10; a leucine at position 11; a valine at position 15; an aspartate at position 17; an arginine at position 18; a valine at position 19; a isoleucine at position 21; an arginine at position 24; a proline at position 39; a lysine at position 41; an alanine at position 42; an aspartate at position 69; a threonine at position 71; a leucine at position 77; a glutamine at position 78; a proline at position 79; a phenylalanine at position 82; a threonine at position 84; and a valine at position 103.

In some embodiments, the Henipavirus antibody comprises: (a) a V_{H} region comprising amino acid sequence SEQ ID NO: 7 and (b) a V_{L} region comprising amino acid sequence SEQ ID NO: 8.

In some embodiments, the Henipavirus antibody comprises: (a) a V_{H} region comprising amino acid sequence SEQ ID NO: 7 and (b) a V_{L} region comprising amino acid sequence SEQ ID NO: 15. (h1F5.2 mAb V_{H} and V_{L} sequences)

In some embodiments, the Henipavirus antibody comprises: (a) a HC comprising amino acid sequence SEQ ID NO: 16 and (b) an LC comprising amino acid sequence SEQ ID NO: 17. (Final MBP1F5 HC and LC sequences)

b. Fc Variants

The Henipavirus antibodies disclosed herein may comprise Fc regions that are modified to alter one or more functional properties of the antibody, such as serum half-life, complement fixation, Fc receptor binding, and/or ADCC. Accordingly, an Fc region can comprise additional mutations to increase or decrease effector functions, i.e., the ability to induce certain biological functions upon binding to an Fc receptor expressed on an immune cell. Immune cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. An Fc region may be modified by conservative amino acid substitution. In some embodiments, the Fc region has at least 1 or 2 conservative substitutions, or at least 50% conservative substitutions. In some embodiments, all the substitutions are conservative substitutions.

In some embodiments, a Henipavirus antibody disclosed herein comprises an Fc amino acid substitution to increase its serum half-life. Such modification may be included in one or both Fc domains of the subject antibody. Examples of substitutions in an Fc region that increase the serum half-life of an antibody include, e.g., M252Y/S254T/T256E, T250Q/M428L, N434A, N434H, T307A/E380A/N434A, M428L/N434S, M252Y/M428L, D259I/V308F, N434S, V308W, V308Y, M430L, N436A, and V308F. In some embodiments, the substitutions in the Fc region include one or both of M430L and N436A. Descriptions of amino acid mutations in an Fc region that can increase the serum half-life of an antibody can be found in, e.g., 0 et al., MAbs. 26:1-10, 2019; Booth et al., MAbs. 10(7):1098-1110, 2018; and Dall'Acqua et al., J Biol Chem. 281(33):23514-24, 2006. In some embodiments, the Fc region mutations are M430L and/or N436A.

In some embodiments, an Fc region can include additional modifications that modulate effector function. Examples of Fc region amino acid mutations that modulate an effector function include, but are not limited to, one or more substitutions at positions 228, 233, 234, 235, 236, 237, 238, 239, 243, 265, 269, 270, 297, 298, 318, 326, 327, 329, 330, 331, 332, 333, and 334 (EU numbering scheme) of an Fc region.

Illustrative substitutions that decrease effector functions include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions that decrease effector functions include S228P, E233P, L235E, N297A, N297D, and P331S. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, to decrease effectors functions. Examples of substitutions that increase effector functions include, e.g., E333A, K326W/E333S, S239D/1332E/G236A, S239D/A330L/1332E, G236A/S239D/A330L/1332E, F243L, G236A, and S298A/E333A/K334A. In some embodiments, the Fc mutations include P329G, L234A, L235A, or a combination thereof. Descriptions of amino acid mutations in an Fc region that can increase or decrease effector functions can be found in, e.g., Wang et al., Protein Cell. 9(1): 63-73, 2018; Saunders, Front Immunol. June 7, eCollection, 2019; Kellner et al., Transfus Med Hemother. 44(5): 327-336, 2017; and Lo et al., J Biol Chem. 292(9):3900-3908, 2017.

In some embodiments, an Fc region may have altered glycosylation that increases the ability of the antibody to recruit NK cells and/or increase ADCC. In some embodiments, the Fc region comprises glycan containing no fucose (i.e., the Fc region is afucosylated). Afucosylated antibodies can be produced using cell lines that express a heterologous enzyme that depletes the fucose pool inside the cell (e.g., GlymaxX® by ProBioGen AG, Berlin, Germany). Non-fucosylated antibodies can also be produced using a host cell line in which the endogenous α-1,6-fucosyltransferase (FUT8) gene is deleted. See Satoh, M. et al., “Non-fucosylated therapeutic antibodies as next-generation therapeutic antibodies,” Expert Opinion on Biological Therapy, 6:11, 1161-1173, DOI: 10.1517/14712598.6.11.1161.

c. Epitope

The Henipavirus antibodies of the present disclosure bind to one or more epitopes on the FC region of the F glycoprotein of Henipavirus. F glycoprotein is synthesized as a F₀ precursor and subsequently cleaved by cathepsin L to produce the disulfide-linked F1 and F2 subunits. In some embodiments, the F₀ is the HeV F₀ (Uniprot ID 089342). In some embodiments, the F₀ is the NiV F₀ (Uniprot ID Q9IH63). F₀ can exist in at least three known conformational states: pre-fusion native state, pre-hairpin intermediate state, and post-fusion hairpin state. In some embodiments, the Henipavirus antibody binds to F₀ in its pre-fusion native state.

Pre-fusion F glycoprotein has been used in the design of two licensed respiratory syncytial virus (RSV) vaccines (Abrysvo: Pfizer; Arexvy: GSK), and as the epitope for a mAb immunoprophylactic (nirsevimab; AstraZeneca). In the case of Henipavirus, cryo-electron microscopy structures and membrane fusion assays indicate that the 12B2 and 1F5 antibodies stabilize F glycoprotein in the pre-fusion state, which is the conformation found at the viral surface before infection. Stabilization of the pre-fusion state prevents the structural rearrangements that lead to membrane fusion between the virus and the host cell. Dang et al., Nature Structural and Molecular Biology (2021).

In some embodiments, the Henipavirus antibody binds to one or more epitopes located on a pre-fusion F glycoprotein. In some embodiments, these epitopes are conserved between NiV, NiV_{B}, NiV_{M}, and/or HeV. In some embodiments, the epitopes are quaternary epitopes of the pre-fusion F₀. In some embodiments, the Henipavirus antibody locks F₀ in its pre-fusion conformation after the antibody binds to its epitope. In some embodiments, the Henipavirus antibody neutralizes NiV, NiV_{B}, NiV_{M}, and/or HeV upon binding its epitope. In some embodiments, the Henipavirus antibody inhibits NiV, NiV_{B}, NiV_{M}, and/or HeV from entering the host cell.

d. Antibody Formats

In some embodiments, a Henipavirus antibody of the present disclosure that is administered to a subject (e.g., a mammal, a human) is an IgG of the IgG1 subclass. In some embodiments, such an antibody is an IgG of the IgG2, IgG3, or IgG4 subclass. In some embodiments, such an antibody is an IgM. In some embodiments, such an antibody has a lambda light chain constant region. In some embodiments, such an antibody has a kappa light chain constant region.

In some embodiments, a Henipavirus antibody of the present disclosure is in a monovalent format. In some embodiments, the Henipavirus antibody is in a fragment format, e.g., a Fv, Fab, Fab′, scFv, diabody, or F(ab′)2 fragment.

Furthermore, in some embodiments, a Henipavirus antibody of the present disclosure may be chemically modified (e.g., one or more chemical moieties can be attached to the antibody) or be modified, e.g., produced in cell lines and/or in cell culture conditions to alter its glycosylation (e.g., hypofucosylation, afucosylation, or increased sialylation), to alter one or more functional properties of the antibody. For example, the antibody can be linked to one of a variety of polymers, for example, polyethylene glycol. In some embodiments, an antibody may comprise mutations to facilitate linkage to a chemical moiety and/or to alter residues that are subject to post-translational modifications, e.g., glycosylation.

In some embodiments, a Henipavirus antibody of the present disclosure is employed in a bispecific or multi-specific format, e.g., a tri-specific format. For example, in some embodiments, the antibody may be incorporated into a bispecific or multi-specific antibody that comprises a further binding domain that binds to the same or a different antigen.

There are a variety of possible formats that can be used in bispecific or multi-specific antibodies. The formats can vary elements such as the number of binding arms, the format of each binding arm (e.g., Fab, scFv, scFab, or V_{H}-only), the number of antigen binding domains present on the binding arms, the connectivity and geometry of each arm with respect to each other, the presence or absence of an Fc domain, the Ig class (e.g., IgG or IgM), the Fc subclass (e.g., hIgG1, hIgG2, or hIgG4), and any mutations to the Fc (e.g., mutations to reduce or increase effector function or extend serum half-life). Also see Speiss, et al., Alternative Molecular Formats and Therapeutic Applications for Bispecific Antibodies, Mol Immunol, 67, 95-106 (2015) for examples of bispecific and multispecific formats.

### 4. Antibody Conjugates and Co-Stimulatory Agents

In a further aspect, a Henipavirus antibody of the present disclosure may be conjugated or linked to therapeutic, imaging/detectable moieties, or enzymes. For example, the antibody may be conjugated to a detectable marker, a cytotoxic agent, an immunomodulating agent, an imaging agent, a therapeutic agent, an oligonucleotide, or an enzyme. Methods for conjugating or linking antibodies to a desired molecule are well known in the art. The moiety may be linked to the antibody covalently or by non-covalent linkages.

In some embodiments, the Henipavirus antibody is conjugated, either directly or via a cleavable or non-cleavable linker, to a cytotoxic moiety or other moiety that inhibits cell proliferation. In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracindione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc 1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis. In some embodiments, the antibody is conjugated to a drug such as a topoisomerase inhibitor, e.g., a topoisomerase I inhibitor.

In some embodiments, the antibody may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### 5. Animal Models of Disease

Development and evaluation of medical countermeasures requires an animal model that accurately recapitulates human disease to predict efficacy. Animal models are used for evaluating therapeutic candidates during outbreaks, like the September 2023 NiV outbreak in Kerala India in which m102.4 was deployed. Various animal models, including mice, guinea pigs, hamsters, and ferrets, as well as cats, pigs, and multiple monkey species (including squirrel monkeys or New World monkeys and African green monkeys (AGMs; grivet monkey; Chlorocebus aethiops)) may be used as animal models of Henipavirus infection.

Of the various animal models that have been described to date (Geisbert, Feldmann et al., 2012, Geisbert, Mire et al., 2014, Mire, Satterfield et al., 2016), non-human primate models (NHPs) have been found to most accurately reflect human disease (Mire, Satterfield et al., 2023). AGMs have been compared to cynomolgus macaques and proposed to be a model host of lethal disease (Prasad, Woolsey et al., 2020) in which disease is mild and non-lethal. Furthermore, NiV_{B} has been demonstrated to be more pathogenic in AGM than NiV_{M} (Mire, Satterfield et al., 2016), suggesting NiV_{B} may be a more suitable strain for countermeasure development.

In some embodiments, hamsters and/or AGMs are used to assess Henipavirus disease and characterize a Henipavirus antibody of the present disclosure.

Animal models may be exposed to viral challenges via a number of administration routes. In some NHP models, intratracheal and/or intranasal administration or small particle aerosol challenge was used (Geisbert, Daddario-DiCaprio et al., 2010, Geisbert, Mire et al., 2014, Mire, Satterfield et al., 2016, Hammoud, Lentz et al., 2018). In some embodiments, viral challenges were administered intranasally and/or intratracheally.

The LMA™ mucosal atomization device (MAD®) may also be used to administer the viral challenges. By delivering atomized particles that range in size from 30 to 100 μm, LMA™ MAD® can represent potential human droplet exposure to a virus (Geisbert, Borisevich et al., 2020) This exposure is consistent with the size of droplets exhaled by humans when coughing or sneezing (Gralton, Tovey et al., 2011), a potential route of person-to-person transmission of Henipavirus. The LMA™ MAD® has been used successfully to deliver viral challenges of Ebola (Alfson, Avena et al., 2017) and severe acute respiratory syndrome coronavirus 2 (SARS-CoV-2) (Cross, Agans et al., 2020) viruses in non-human primate (NHP) models. In some embodiments, AGMs were exposed to Henipavirus intranasally via a LMA™ MAD® as previously described (Geisbert, Borisevich et al., 2020). In some embodiments, the Henipavirus is NiV, NiV_{B}, NiV_{M}, or HeV.

### 6. Antibody Expression and Purification, Nucleic Acids, Vectors, and Cells

Henipavirus antibodies as disclosed herein are commonly produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the invention, provided herein are recombinant nucleic acids encoding a V_{H} and/or V_{L} region, or fragment thereof, of any of the antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector can be replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing a Henipavirus antibody as described herein include both prokaryotic or eukaryotic cells. For example, a Henipavirus antibody may be produced in bacteria, in particular when glycosylation and Fc effector function are not needed. After expression, the antibody may be isolated from the bacterial cell paste in a soluble fraction and can be further purified. Thus, as used herein, an “isolated” antibody may refer to an antibody that has been separated or purified from its natural environment or the environment where it was expressed, e.g., cytoplasm, cell membrane, cell extract, and cell lysate. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing a Henipavirus antibody of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59, 1977; baby hamster kidney cells (BHK); mouse sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express an antibodies. Other useful mammalian host cell lines include Chinese hamster ovary (CHO) cells, including DHFR-CHO cells (Urlaub et al., Proc. Natl. Acad. Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268, 2003.

In some embodiments, a Henipavirus antibody of the present disclosure is produced by a CHO cell line, e.g., ExpiCHO™, ExpiCHO-S™, CHOZN®, and CHO-K1 cell lines. One or more expression plasmids can be introduced that encode heavy and light chain sequences. In some embodiments, the CHOZN® GS-/-ZFN-modified Chinese hamster ovary (CHO) cell line (Sigma Aldrich) was transfected with an expression vector containing the humanized IgG1 gamma and kappa 1F5 structural genes. The resultant clonal cell line was isolated from the transfected stable pool using industry standard screening and selection methods.

A host cell transfected with an expression vector encoding a Henipavirus antibody of the present disclosure, or fragment thereof, can be cultured under appropriate conditions to allow expression of the polypeptide to occur. In some embodiments, a method of producing a Henipavirus antibody includes culturing a host cell under conditions in which the polynucleotide encoding the heavy chain and the polynucleotide encoding the light chain are expressed. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, a Henipavirus antibody of the present disclosure can be produced by in vitro synthesis (see, e.g., Sutro Biopharma biochemical protein synthesis platform).

### 7. Pharmaceutical Compositions

The Henipavirus antibodies disclosed herein are suitable for administration in vitro or in vivo. In some embodiments, pharmaceutical compositions comprise a Henipavirus antibody of the present disclosure and a pharmaceutically acceptable carrier (excipient). A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Suitable formulations for use in the present invention are found, e.g., in Remington: The Science and Practice of Pharmacy, 21st Edition, Philadelphia, PA. Lippincott Williams & Wilkins, 2005.

The pharmaceutical compositions may be administered to a subject (e.g., a mammal, a human) in an amount sufficient to induce an immune response. In some embodiments, the pharmaceutical composition may cure or at least partially arrest the disease or symptoms of a Henipavirus infection and its complications. The pharmaceutical compositions may also be administered to a subject to provide a prophylactic benefit. An amount adequate to accomplish any of the above benefits is defined as a “therapeutically effective dose.” A therapeutically effective dose is determined by monitoring a patient's response to therapy. Typical benchmarks indicative of a therapeutically effective dose includes the amelioration of symptoms of the disease in the patient. Amounts effective for this use will depend upon the severity of the disease and the general state of the patient's health, including other factors such as age, weight, gender, administration route, and the like, single or multiple administrations of the antibody may be administered depending on the dosage and frequency as required and tolerated by the patient. In any event, the methods provide a sufficient quantity of Henipavirus antibody to effectively treat the subject.

The pharmaceutical compositions can be administered by any suitable means, including, for example, parenteral, intrapulmonary, and intranasal, administration, as well as local administration. Parenteral infusions include intratracheal, intramuscular, intravenous, intraarterial, intraperitoneal, or subcutaneous administration. In some embodiments, the antibody may be administered by LMA™ MAD®. In some embodiments, the antibody may be administered by insufflation.

In some embodiments, the pharmaceutical composition is administered to a patient after the patient is exposed to Henipavirus and/or after the patient is diagnosed with a Henipavirus infection. In some embodiments, the pharmaceutical composition is administered to the patient as a post-exposure prophylaxis to prevent the Henipavirus infection from developing or progressing. In some embodiments, the pharmaceutical composition is administered to the patient after the patient is exposed to Henipavirus and before the onset of symptoms of a Henipavirus infection. In many cases, symptoms of a Henipavirus infection may include fever, headache, muscle aches, myalgia, cough, fatigue, vomiting, sore throat, dizziness, drowsiness, confusion, disorientation, seizure, encephalitis, confusion, abnormal reflexes, respiratory symptoms, shallow breathing, ataxia, facial swelling, depression, frothing of saliva, nasal discharge, influenza-like symptoms, anorexia, and/or coma. In some embodiments, the pharmaceutical composition is administered to a subject prior to exposure to a Henipavirus as a pre-exposure prophylaxis (PrEP). In some embodiments, subject is diagnosed as not having a Henipavirus infection and is administered the pharmaceutical composition as a PrEP.

In some embodiments, a therapeutically effective dose of a pharmaceutical composition may vary from about 0.01 mg/kg to about 300 mg/kg, preferably from about 0.1 mg/kg to about 50 mg/kg, most preferably from about 0.2 mg/kg to about 10 mg/kg. In some embodiments, the dose can be about 20, about 10, about 5, about 2.5, or about 1 mg/kg, or any intervening dose between about 1 mg/kg and 20 mg/kg. In some embodiments, the dose is a flat dose that may vary from about 100 to 5000 mg. As used herein, the term “flat dose” refers to the a dose to be administered to any individual without regard to the individual's weight.

In some embodiments, the pharmaceutical composition is administered in one or more doses. In some embodiments, the pharmaceutical composition is administered daily, weekly, every two weeks, monthly, every two to twelve months, for example, every two months, every three months, every four months, every five months, every six months, every seven months, every eight months, every nine months, every ten months, every eleven months, or every twelve months, for a period of time. In some embodiments, pharmaceutical composition is administered for 2 to 10 or more consecutive days. In some embodiments, the pharmaceutical composition is administered after 1 to 4 weeks or after 1 to 6 months. In some embodiments, the pharmaceutical composition is administered to a subject at least 1, 2, 3, 4, 5, 6, 7, 8, 9, or 10 times.

### 8. Methods of Use

a. Methods of Treatment and Prophylaxis

The Henipavirus antibodies disclosed herein can be used to induce an immune response, and therefore be used as a prophylactic agent or a therapeutic agent to treat one or more diseases or symptoms associated with an infectious disease cause by any member of the Henipavirus genus. In some embodiments, the Henipavirus infectious disease is caused by NiV, NiV_{B}, NiV_{M}, or HeV. In some embodiments, the antibody is a bispecific or multispecific antibody described herein. In some embodiments, the Henipavirus antibody is hu1F5, hu12B2, or MBP1F5, or a variant thereof.

The Henipavirus antibodies can be used to treat one or more diseases or symptoms caused by one or more members of the Henipavirus genus. In some embodiments, the Henipavirus is NiV, NiV_{B}, NiV_{M}, or HeV. In some embodiments, a subject (e.g., a mammal, a human) who can benefit from the treatment of the Henipavirus antibody has one or more symptoms that may be associated with a Henipavirus infection, e.g., fever, headache, cough, sore throat, difficulty breathing, vomiting, disorientation, drowsiness, confusion, seizures, coma, encephalitis, convulsions, and/or personality changes.

In some embodiments, the Henipavirus antibodies disclosed herein neutralizes one or more members of the Henipavirus genus. The epitopes on the Henipavirus F glycoprotein that the Henipavirus antibodies of the present disclosure can bind to are discussed above. Based on sequence homology between F glycoproteins of different Henipavirus genus members, one of ordinary skill in the art can determine whether a Henipavirus antibody disclosed herein can bind to a F glycoprotein that is expressed by any member of the Henipavirus genus. Similarly, based on sequence homology between F glycoproteins, one of ordinary skill in the art can determine whether a Henipavirus antibody disclosed herein can neutralize any member of the Henipavirus genus. In some embodiments, the Henipavirus antibody neutralizes NiV, NiV_{B}, NiV_{M}, or HeV. In some embodiments, the Henipavirus antibody prevents NiV, NiV_{B}, NiV_{M}, or HeV from entering the cell. In some embodiments, if the F glycoprotein from the first Henipavirus and the F glycoprotein from the second Henipavirus share at least 65%, at least at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity over the full length of at least one of the two F glycoproteins, a Henipavirus antibody can bind to the F glycoprotein on a first Henipavirus is expected to be able to bind to the F glycoprotein on a second Henipavirus, and a Henipavirus antibody can neutralize the first Henipavirus is also expected to be able to neutralize the second Henipavirus. The binding activity and neutralizing activity can be readily confirmed by using methods well known in the art and/or those disclosed in this application.

The Henipavirus antibodies disclosed herein can also be used as a prophylaxis agent. In some embodiments, the Henipavirus antibody is a pre-exposure prophylaxis (PrEP) that provides protect against infection prior to a subject's exposure to a Henipavirus. In some embodiments, the Henipavirus antibody is a post-exposure prophylaxis that is administered to a subject that has been exposed to a Henipavirus but the subject has not yet experienced the onset of symptoms of Henipavirus. In some embodiments, the post-exposure prophylaxis is administered to prevent the progression or development of the disease or symptoms of the Henipavirus in the subject. In some embodiments, the Henipavirus is NiV, NiV_{B}, NiV_{M}, Cedar henipavirus (CedV), Ghanaian bat henipavirus (Kumasi virus; KV), Langya henipavirus (LayV), Mojiang henipavirus (MojV), or HeV.

In some embodiments, the Henipavirus is carried by Pteropus bats, squirrel monkeys, New World monkeys, AGMs, mice, guinea pigs, hamsters, and ferrets, cats, and pigs. In some embodiments, the Henipavirus antibody neutralizes one or more viruses carried by Pteropus bats, squirrel monkeys, New World monkeys, AGMs, mice, guinea pigs, hamsters, and ferrets, cats, and pigs.

The Henipavirus antibodies disclosed herein are provided in a solution suitable for administration to a subject, such as a sterile isotonic aqueous solution. The Henipavirus antibodies are dissolved or suspended at a suitable concentration in pharmaceutical compositions as discussed herein.

b. Combination Therapy

In some embodiments, one or more of Henipavirus antibodies of the present disclosure of may be administered with one or more additional antibodies. A Henipavirus antibody of may be administered with one or more additional therapeutic agents, e.g., an immunostimulatory agent, an immunotherapeutic agent. In some embodiments, a Henipavirus antibody of the present invention may be administered with an agent, e.g., a corticosteroid, that mitigates side-effects resulting from stimulation of the immune system.

In accordance with the present disclosure, an additional therapeutic agent that is administered with a Henipavirus antibody disclosed herein can be administered prior to administration of the Henipavirus antibody or after administration of the Henipavirus antibody. In some embodiments, the Henipavirus antibody may be administered at the same time as the additional therapeutic agent. In some embodiments, the Henipavirus antibody and an additional therapeutic agent described above can be administered following the same or different dosing regimens. In some embodiments, the Henipavirus antibody and the therapeutic agent are administered sequentially in any order during the entire treatment period or portions thereof. In some embodiments, the Henipavirus antibody and the therapeutic agent are administered simultaneously or approximately simultaneously (e.g., within about 1, 5, 10, 15, 20, or 30 minutes of each other). In still other embodiments, the therapeutic agent may be administered 1, 2, 3, 4, 5, 6, 7, 8, 9, 10, or more days before the administration of the Henipavirus epitope targeting antibody. In still other embodiments, the therapeutic agent may be administered 1, 2, 3, 4, 5, 6, 7, 8, 9, 10, or more days after the administration of the Henipavirus epitope targeting antibody.

c. Methods of Diagnosis

The Henipavirus antibodies disclosed herein can also be used for diagnosing a patient having a Henipavirus infection. In one aspect, the method comprises contacting a biological sample (e.g., nasal mucus, saliva, whole blood, serum, plasma, cerebrospinal fluid, urine) from a subject (e.g., a mammal, a human) with an antibody disclosed herein and detecting binding of the antibody to the biological sample. In some cases, the antibodies are conjugated to a detectable label that produces fluorescent, luminescent, or colorimetric signals, and detecting the signal from the label indicates a subject is suitable for treatment with a Henipavirus antibody disclosed herein.

### 9. Embodiments

Embodiment 1. An isolated humanized antibody that binds to Henipavirus F glycoprotein, wherein the isolated humanized antibody comprises: (i) a heavy chain variable region (VH) with an amino acid substitution mutation at position 25 relative to a sequence of SEQ ID NO: 9, optionally wherein the VH amino acid substitution mutation is T25S relative SEQ ID NO: 9; and/or (ii) a light chain variable region (VL) with an amino acid substitution mutation at position 24 relative to a sequence of SEQ ID NO: 10, optionally wherein the VL amino acid substitution mutation is S24R relative to SEQ ID NO: 10.

Embodiment 2. The isolated humanized antibody of embodiment 1, wherein the isolated humanized antibody binds to an epitope located on a pre-fusion form of the Henipavirus F glycoprotein.

Embodiment 3. The isolated humanized antibody of embodiment 2, wherein the Henipavirus F glycoprotein is a Nipah virus (NiV) F glycoprotein or a Hendra virus (HeV) F glycoprotein.

Embodiment 4. The isolated humanized antibody of any one of the preceding embodiments, wherein the isolated antibody can treat Nipah virus (NiV), Bangladesh strain of NiV (NiVB), Malaysia strain of NiV (NiVM), Cedar henipavirus (CedV), Ghanaian bat henipavirus (Kumasi virus; KV), Langya henipavirus (LayV), Mojiang henipavirus (MojV), and Hendra virus (HeV).

Embodiment 5. The isolated humanized antibody of any one of the preceding embodiments, wherein the isolated humanized antibody comprises: (i) a heavy chain complementarity-determining region (HCDR) comprising: (a) an HCDR1 comprising a sequence of SEQ ID NO: 1 (SGYSITSDYYW); (b) an HCDR2 comprising a sequence of SEQ ID NO: 2 (VTYDGSN); and (c) an HCDR3 comprising a sequence of SEQ ID NO: 3 (RFGSSYWAMDYW); and (ii) a light chain complementarity-determining region (LCDR) comprising: (a) an LCDR1 comprising a sequence of SEQ ID NO: 4 (RASSSVSYMH); (b) an LCDR2 comprising a sequence of SEQ ID NO: 5 (STSNLAS); and (c) an LCDR3 comprising a sequence of SEQ ID NO: 6 (HQWYSYPWT).

Embodiment 6. The isolated humanized antibody of any one of embodiments 1-4, wherein the isolated humanized antibody comprises: (i) an HCDR1 comprising a sequence of SEQ ID NO: 1 (SGYSITSDYYW); (ii) an HCDR2 comprising a sequence of SEQ ID NO: 2 (VTYDGSN); (iii) an HCDR3 comprising a sequence of SEQ ID NO: 3 (RFGSSYWAMDYW); (iv) an LCDR1 comprising a sequence of SEQ ID NO: 4 (RASSSVSYMH); (v) an LCDR2 comprising a sequence of SEQ ID NO: 5 (STSNLAS); and (vi) an LCDR3 comprising a sequence of SEQ ID NO: 6 (HQWYSYPWT).

Embodiment 7. The isolated humanized antibody of any one of the preceding embodiments, wherein the VH comprises an amino acid sequence having at least 95% identity to the sequence of SEQ ID NO: 7, 9, or 15, and the VL sequence comprises an amino sequence having at 95% identity to the sequence of SEQ ID NO: 8 or 10.

Embodiment 8. The isolated humanized antibody of any one of embodiments 1-6, wherein (i) the VH comprises at least 95% identity to the sequence of SEQ ID NO: 7, 9, or 15; and (a) an HCDR1 comprising the sequence of SEQ ID NO: 1; (b) an HCDR2 comprising the sequence of SEQ ID NO: 2; and (c) an HCDR3 comprising the sequence of SEQ ID NO: 3; and (ii) the VL comprises at least 95% identity to the sequence of SEQ ID NO: 8 or 10, and (a) an LCDR1 comprising the sequence of SEQ ID NO: 4; (b) an LCDR2 comprising the sequence of SEQ ID NO: 5; and (c) an LCDR3 comprising the sequence of SEQ ID NO: 6.

Embodiment 9. The isolated humanized antibody of any one of embodiments 1-8, wherein at least 1 or 2 of the substitutions are conservative substitutions, at least 50% of the substitutions are conservative substitutions, or all of the substitutions are conservative substitutions.

Embodiment 10. The isolated humanized antibody of any one embodiments 1-9, wherein (i) the VH sequence comprises the sequence of SEQ ID NO: 7 and (ii) the VL sequence comprises the sequence of SEQ ID NO: 8.

Embodiment 11. The isolated humanized antibody of any one embodiments 1-9, wherein (i) the VH sequence comprises the sequence of SEQ ID NO: 15 and (ii) the VL sequence comprises the sequence of SEQ ID NO: 8.

Embodiment 12. The isolated humanized antibody of any one embodiments 1-9 and 11, wherein (i) the HC sequence comprises an amino acid sequence having at least 95% identity to the sequence of SEQ ID NO: 16, and (ii) the LC sequence comprises an amino acid sequence having at least 95% identity to the sequence of SEQ ID NO: 17.

Embodiment 13. The isolated humanized antibody of embodiment 12, wherein (i) the HC sequence comprises the sequence of SEQ ID NO: 16, and (ii) the LC sequence comprises the sequence of SEQ ID NO: 17.

Embodiment 14. The isolated humanized antibody of any one of the preceding embodiments, wherein the isolated humanized antibody further comprises at least one Fc mutation, wherein the at least one Fc mutation confers increased half-life.

Embodiment 15. The isolated humanized antibody of embodiment 14, wherein the at least one Fc mutation is selected from the group consisting of M430L and N436A.

Embodiment 16. A humanized antibody that binds to the same epitope as the isolated antibody of any one of embodiments 1-15.

Embodiment 17. A humanized antibody that competes for binding with the isolated humanized antibody of embodiments 1-15.

Embodiment 18. The isolated antibody of any one of embodiments 1-15 or the humanized antibody of embodiment 16 or 17, wherein the antibody is a non-natural antibody.

Embodiment 19. A recombinant nucleic acid encoding the antibody according to any one of embodiments 1-15 or the humanized antibody of embodiment 16 or 17.

Embodiment 20. An expression vector comprising a polynucleotide encoding a VH sequence and/or a VL sequence of the antibody of any one of embodiment 1-15 or the humanized antibody of embodiment 16 or 17.

Embodiment 21. A host cell that comprises an expression vector of embodiment 20.

Embodiment 22. A method of producing a humanized antibody, the method comprising culturing a host cell of embodiment 21 under conditions in which one or more polynucleotides encoding a heavy chain and/or a light chain are expressed.

Embodiment 23. A polypeptide comprising (i) a VH sequence of SEQ ID NO: 7 or having at least 70% amino acid sequence identity to a sequence of SEQ ID NO: 7 and/or (ii) a VL sequence of SEQ ID NO: 8 having at least 70% amino acid sequence identity to a sequence of SEQ ID NO: 8.

Embodiment 24. A polypeptide comprising (i) a VH sequence of SEQ ID NO: 15 or having at least 70% amino acid sequence identity to a sequence of SEQ ID NO: 15 and/or (ii) a VL sequence of SEQ ID NO: 8 having at least 70% amino acid sequence identity to a sequence of SEQ ID NO: 8.

Embodiment 25. A polypeptide comprising (i) a HC sequence of SEQ ID NO: 16 or having at least 70% amino acid sequence identity to a sequence of SEQ ID NO: 16 and/or (ii) an LC sequence of SEQ ID NO: 17 having at least 70% amino acid sequence identity to a sequence of SEQ ID NO: 17.

Embodiment 26. A pharmaceutical composition comprising the humanized antibody of any one of embodiments 1-18 and a pharmaceutically acceptable carrier.

Embodiment 27. The pharmaceutical composition of embodiment 26, wherein the pharmaceutical composition comprises two or more different humanized antibodies of any one of embodiments 1-18.

Embodiment 28. A method of inducing an immune response and/or treating an infectious disease, the method comprising administering an effective amount of a pharmaceutical composition of embodiment 26 or 27 to a subject in need thereof.

Embodiment 29. The method of embodiment 28, wherein the administering step occurs after the subject has been exposed to a Henipavirus that causes the infectious disease and before onset of symptoms of the infectious disease in the subject.

Embodiment 30. The method of embodiment 28, wherein the administering step occurs before the subject is exposed to a Henipavirus that causes the infectious disease.

Embodiment 31. The method of any one of embodiments 28-30, wherein the Henipavirus is NiV, NiV_{B}, NiV_{M}, CedV, KV, LayV, MojV, and/or HeV.

### EXAMPLES

Overview—The following Examples discuss the efficacy conferred by humanized anti-F glycoprotein neutralizing monoclonal antibodies (mAbs) in hamster and African green monkey (AGM) models; the AGM model closely represents Henipavirus disease in humans (Mire, Satterfield et al., 2023).

Hu1F5 and hu12B2 are humanized mAbs that recognize two unique pre-fusion-specific quaternary epitopes that are conserved between HeV and NiV; Hu1F5 and hu12B2 lock F glycoprotein in its pre-fusion conformation (Dang, Cross et al., 2021). In order to humanize the mouse-derived antibodies 1F5 and 12B2, the mouse variable fragment (Fv) sequences for each heavy chain (HC) and light chain (LC) were each blasted separately against human germline antibody genes using the IgGBLAST tool (ncbi.nlm.nih.gov/igblast/). The closest human germline matching the 1F5 and 12B2 murine sequences for the HC or LC was used as a sequence template for the humanization of each respective Fv. The murine sequence for the Fv was then used to generate a 3D structural model within the Molecular Operating Environment (MOE), a molecular modeling software package (chemcomp.com). With the structural model and germline analysis available, a structure-guided resurfacing approach was used to humanize 1F5 and 12B2.

One humanized 12B2 variant was produced, hu12B2, and two humanized 1F5 variants were produced, h1F5.1 and h1F5.2. Of the two humanized 1F5 variants, h1F5.2 was produced with a more conservative humanization approach wherein mutations to human germline were not made within the predicted binding interface. After humanization was complete, it was confirmed that no modification of residues within 4 Å of the 1F5 antigen-binding interface had occurred (see mouse mAb structures as discussed in Dang et al 2021; (5)). The two humanized variants, h1F5.1 and h1F5.2, were cloned, expressed, purified, and tested for binding to soluble Henipavirus F protein. h1F5.2 displayed a more favorable profile overall during these steps and was selected for advancement to hamster challenge studies. h1F5.2 is also generally referred to as hu1F5 in this disclosure.

Hu1F5 and hu12B2 were compared in a hamster model for NiV and were found to neutralize NiV_{B}, NiV_{M} and HeV (Dang, Cross et al., 2021). The hamster model is reasonable for initial screening of drug candidates but does not recapitulate human disease as well as AGM models (Mire, Satterfield et al., 2023). The survival difference between hu1F5 (100%) and hu12B2 (60%) treated animals did not differ statistically.

hu1F5 was selected for comparison in the AGM model to m102.4, the mAb currently used under compassionate use for NiV and HeV post-exposure prophylaxis. It has been suggested that escape mutations around F glycoprotein specific mAbs would hinder viral fitness and virulence. Dang, H. V., et al., An antibody against the F glycoprotein inhibits Nipah and Hendra virus infections. Nature Structural & Molecular Biology, 2019. 26(10): p. 980-987. Escape mutations can arise due to genetic changes in the pathogen that help the pathogen evade detection and elimination by the host immune system and survive. No escape mutants were isolated for hu1F5.

Compared to m102.4, an anti-G glycoprotein human mAb, hu1F5 was superior in rescuing AGMs from NiV disease. m102.4 has been used in the clinic for post-exposure prophylaxis of NiV. Thus, the Examples demonstrate that hu1F5 is an improvement over m102.4 and is a candidate for continued development as a prophylactic, post-exposure prophylactic, and therapeutic mAb in humans.

### Example 1—Materials and Methods

Production of hu1F5 and hu12B2—For hamster studies, hu12B2 and hu1F5 were transiently expressed in ExpiCHO cells (Thermo Fisher Scientific) following the high titer protocol for CHO Expifectamine-based expression (Thermo Fisher Scientific). Cultures were spun down 9-10 days after transfection. The supernatant was filtered and loaded on to a HiTrap MabSelect™ SuRe™ affinity column (Cytiva) using an AKTA pure fast protein liquid chromatography (FPLC) system. The column was washed with 10 column volumes of phosphate buffered saline pH 7.2 and antibodies were eluted with Pierce IgG elution buffer (Thermo Fisher Scientific). Fractions containing antibody were combined and neutralized to ˜pH 7 with 1 M Tris pH 7.8. For the AGM studies, CHOZN® cells (Sigma) stably expressing hu1F5 were generated as previously described (Bornholdt, Herbert et al., (2019). “A Two-Antibody Pan-Ebolavirus Cocktail Confers Broad Therapeutic Protection in Ferrets and Nonhuman Primates.” Cell Host Microbe 25(1): 49-58 e45). Briefly, a dual-promoter plasmid containing expression cassettes for the heavy and light chains of the mAb was transfected into the CHOZN® cell line with MSX selection beginning 24 h post transfection. Upon recovery of the cells (˜3 weeks), the enriched pool of CHOZN® cells was expanded for expression of hu1F5 in BalanCD Growth A medium containing 25 μM MSX. The culture was placed in a shaking CO₂ incubator set at 5% CO₂ and 140 RPM and expanded over the next 14 days to support large scale production of hu1F5 in multiple 5 L shake flasks (Thomson). The cultures were then maintained in batch-mode for 10 days. The supernatant was clarified via filtration and purified using a Cytiva MabSelect™ SuRe™ LX Protein A affinity chromatography column on an AKTA pure 150 system. Hu1F5 was loaded onto the column, washed with HyClone™ 1×PBS, and eluted with 0.1 M acetic acid, pH 3.3. The eluate was immediately neutralized with 2 M Tris base to PH˜7. The neutralized eluate was then diluted 4.7-fold with WFI quality water and purified using a Cytiva Capto™ Q chromatography column in a flow-through mode for endotoxin and host-cell DNA removal. The Capto™ Q flow-through containing the hu1F5 was diafiltered against the formulation buffer (20 mM histidine, 5% sorbitol, pH 6.0) and concentrated to 30.4 mg/mL using a Sartorius 30 kD Sartocon® Slice ECO Hydrosart® cassette on a Sartorius Sartoflow® Smart TFF system. Polysorbate-80 was added to 0.05% after the target mAb concentration was reached and the solution was filter sterilized.

Virus Isolate—The isolate of NiV_{B} used in the study was 200401066, which was obtained from a fatal human case during the outbreak in Rajbari, Bangladesh in 2004 and passaged on Vero-E6 cells twice (Mire, Satterfield et al., 2016). There were four mutations of sufficient frequency to note between the P2 stock of NiV_{B} and the reference sequence GenBank Accession number AY988601.1. Of these, one was non-coding, and the other three led to single amino acid changes: one in the M protein and two in the F glycoprotein (Mire, Satterfield et al., 2016).

Post-exposure prophylaxis in hamsters—Three- to five-week-old Syrian golden hamsters were inoculated with 5×10° PFU NiVe (passage 3) via the intranasal route. At 24 hours post challenge, 5 animals per group were treated with 5 mg/kg 1F5 or 12B2 by intraperitoneal (i.p.) administration. A single cohort of hamsters was treated with combined administration of 2.5 mg/kg each of 1F5 and 12B2 by the same route. A control animal was treated with PBS only. Animals were monitored for 28 days for changes in weight, temperature, and clinical appearance. Animals were humanely euthanized once reaching the defined euthanasia criteria or at the experimental endpoint.

Efficacy in AGMS infected with NiV_{B}—For the first study, thirteen healthy, adult AGMs (6 males and 7 females) from Barbados (Chlorocebus aethiops; PreLabs) were randomized into treatment and control groups using Microsoft Excel. All 13 AGMs were exposed to a target dose of 40,000 PFU of NiV_{B} (actual dose 37,500 PFU) by intranasal (i.n.) administration using the LMA™ MAD® as previously described (Geisbert, Borisevich et al., (2020). “An Intranasal Exposure Model of Lethal Nipah Virus Infection in African Green Monkeys.” J Infect Dis 221 (Suppl 4): S414-S418). At 5 dpe, one experimental group of five AGMs was treated by intravenous (i.v.) administration with 25 mg/kg of m102.4 while a second experimental group of AGMs was treated by i.v. administration with 25 mg/kg of hu1F5. The virus positive control animal was not treated in this study. Surviving animals were euthanized at the predetermined study endpoint on day 35 after NiV infection.

For the second study, four healthy adult AGMs (3 males and 1 female) from Barbados (PreLabs) were randomized into treatment and control groups using Microsoft Excel. All 4 AGMs were exposed to a target dose of 40,000 PFU of NiV_{B} (actual dose 42,000 PFU) by i.n. administration using the LMA™ MAD® as previously described (Geisbert, Borisevich et al., (2020). “An Intranasal Exposure Model of Lethal Nipah Virus Infection in African Green Monkeys.” J Infect Dis 221 (Suppl 4): S414-S418). At 5 dpe, the experimental group of three AGMs was treated by i.v. administration with 10 mg/kg of hu1F5. The virus positive control animal was not treated in this study. Surviving animals were euthanized at the predetermined study endpoint on day 35 after NiV infection.

All animals for both studies were given physical examinations, and blood was collected before vaccination (day 0); and on days 5, 8, 11, 14, 21, 28, and 35 after virus challenge. The AGMs were monitored daily and scored for disease progression with an internal NiV humane endpoint scoring sheet approved by the University of Texas Medical Branch Institutional Animal Care and Use Committee (UTMB IACUC). Scoring criteria was based on parameters such as respiration (0-9), appetite (0-2), activity/appearance (0-9), and neurological signs (0-9). A score > or equal to 9 met euthanasia criteria.

The use of individual controls and comparisons with external controls using the same viral stock and protocols were performed by the same personnel, as has become routine in many high-containment laboratories for highly lethal pathogens and is preferred by many IACUCs. The decision for this study was dictated by budgetary constraints, BSL-4 space limitations, and the University of Texas Medical Branch (UTMB) IACUC.

RNA isolation from NiV_{B}-infected AGMs—On procedure days, 100 μL of blood from K2-EDTA collection tubes was collected prior to centrifugation and added to 600 μL of AVL viral lysis buffer (Qiagen, Germantown, MD; Catalog #19073) with 6 μL carrier RNA (Qiagen, Germantown, MD; Catalog #1017647) for RNA extraction. For tissues, approximately 100 mg was stored in 1 mL RNAlater (Invitrogen, Waltham MA; Catalog #AM7024) for a minimum of 24 hours. RNA was completely removed, and tissues were homogenized in 600 μL RLT buffer and 1% beta-mercaptoethanol (Qiagen, Germantown, MD; Catalog #79216) in a 2 mL cryovial using a tissue lyser (Qiagen, Germantown, MD; Catalog #85600) and 0.2 mm ceramic beads. The tissues sampled included axillary and inguinal lymph nodes, liver, spleen, kidney, adrenal gland, lung, pancreas, urinary bladder, ovary or testis, and eye. All blood samples were inactivated in AVL viral lysis buffer, and tissue samples were homogenized and inactivated in RLT buffer prior to removal from the BSL-4 laboratory. Subsequently, RNA was isolated from blood using the QIAamp® viral RNA kit (Qiagen, Germantown, MD; Catalog #52962), and from tissues using the RNeasy® minikit (Qiagen, Germantown, MD; Catalog #74104) according to the manufacturer's instructions supplied with each kit.

Detection of NiV_{B} load—RNA was isolated from blood or tissues and assessed using primers/probe targeting the middle of the N gene of NiV_{B} for reverse transcriptase quantitative PCR (RT-qPCR) with the probe used being 6FAM-5′-CTGCAGGAGGTGTGCTCACGGAGG-3′-TAMRA (SEQ ID NO: 18) (Life Technologies, Carlsbad, CA) as described previously (Mire, Satterfield et al., 2016). NiV_{B} RNA was detected using the CFX96 detection system (Bio-Rad) in One-step probe RT-qPCR kits (Bio-Rad, Hercules, CA; Catalog #1725070) with the following cycle conditions: 50° C. for 10 minutes, 95° C. for 10 seconds, and 45 cycles of 95° C. for 10 seconds and 57° C. for 30 seconds. Threshold cycle (CT) values representing NiV_{B} genomes were analyzed with CFX Manager Software, and data are presented as GEq. To generate the GEq standard curve, RNA from NiV_{B} challenge stocks was extracted and the number of genomes was calculated using Avogadro's number and the molecular weight of the NiV_{B} genome.

Virus titration was performed by plaque assay with Vero cells from all blood and tissue samples. Briefly, increasing 10-fold dilutions of the samples were adsorbed to Vero cell monolayers in duplicate wells (200 μL); the limit of detection was 25 PFU/mL. To determine circulating viremia, titrations were performed on the plasma fraction of blood collected in K2-EDTA collection tubes.

Hematology and serum biochemistry—Total white blood cell counts, white blood cell differentials, red blood cell counts, platelet counts, hematocrit values, total hemoglobin concentrations, mean cell volumes, mean corpuscular volumes, and mean corpuscular hemoglobin concentrations were analyzed from blood collected in tubes containing EDTA using a laser-based hematologic analyzer (Beckman Coulter). Serum samples were tested for concentrations of albumin, amylase, alanine aminotransferase (ALT), aspartate aminotransferase (AST), alkaline phosphatase (ALP), blood urea nitrogen (BUN), calcium, creatinine (CRE), C-reactive protein (CRP), gamma-glutamyltransferase (GGT), glucose, total protein, and uric acid by using a Piccolo point-of-care analyzer and Biochemistry Panel Plus analyzer discs (Abaxis).

Statistics—The log-rank test was used for analyzing survival data (GraphPad Prism 10 for macOS; version 10.0.1).

### Example 2—Comparison of hu1F5 and hu12B2 in Hamsters Exposed to NiV_{B}

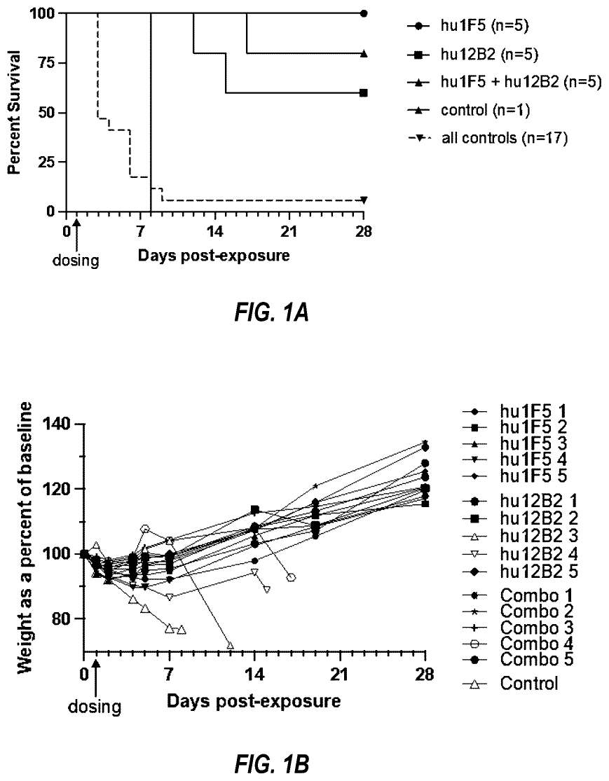

Groups of Syrian golden hamsters (n=5 per group) were exposed intranasally to 5×10⁶ PFU of NiV_{B}. One day after exposure, animals received either PBS (control group) or 5 mg/kg of hu1F5, hu12B2, or hu1F5+hu12B2 (2.5 mg/kg each) intraperitoneally (i.p.). As FIG. 1A illustrates, 100% of animals treated with hu1F5 survived (P=0.03 compared to control by log-rank test) and 60% treated with hu12B2 survived. In the group receiving the two mAbs combined, the survival rate (80%) was in between that of the groups receiving a single mAb, suggesting the superior potency of hu1F5 was diluted out by addition of hu12B2. The control in this study succumbed 8 days post-exposure (dpe) while the cumulative controls (same virus stock, protocol and personnel) had a mean time to death of 4.5 days. All animals experienced weight loss for a minimum of 2-4 dpe with the control animal never experiencing a rebound (FIG. 1B). The three treated animals that succumbed to NiV disease experienced an increase in weight prior to rapid weight loss and before ultimately succumbing. In total, these results suggest that hu1F5 monotherapy was superior in this model and therefore it was selected for comparison to m102.4 in AGMs.

### Example 3—Activity of m102.4 and hu1F5 in AGMs Exposed to NiV_{B}

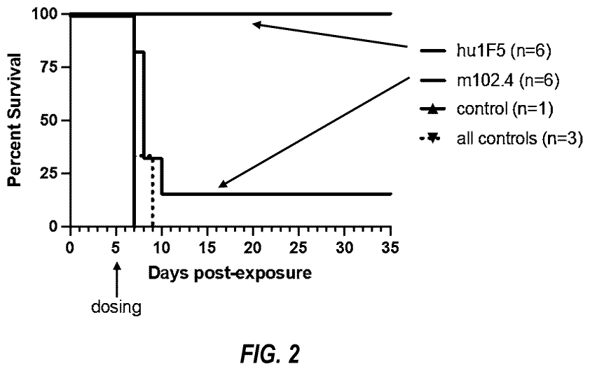

Groups of 6 animals were exposed to 40,000 PFU of NiV_{B} intranasally using MAD and treated 5 days later with a single 25 mg/kg intravenous dose of m102.4 or hu1F5. While only 1 of 6 animals treated with m102.4 survived until the end of the experiment, all 6 AGMs treated with hu1F5 survived (P<0.005 by log-rank test; FIG. 2). In contrast, the contemporaneous untreated control animal succumbed to disease 7 dpe (P=0.01 compared to the hu1F5-treated group) and the untreated external controls (identical route of exposure and viral stock) succumbed 7 and 9 dpe (mean time to death of 7.7 days; n=3; P<0.005 compared to the hu1F5 group).

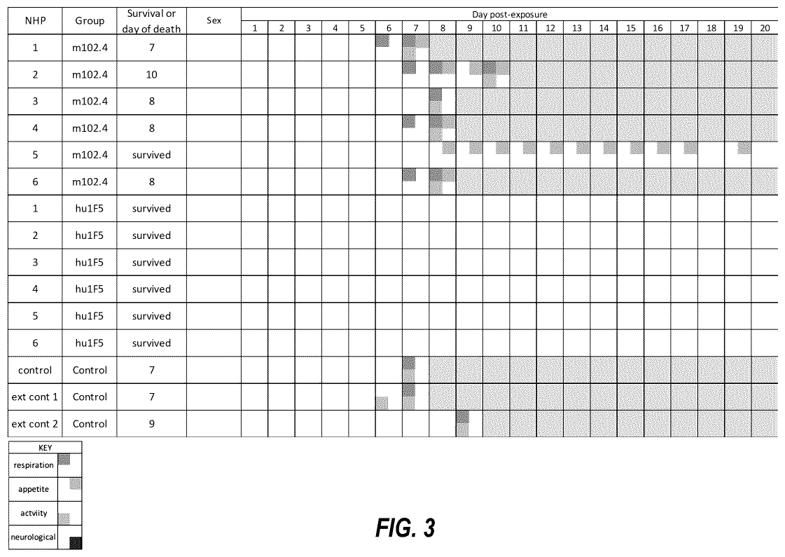

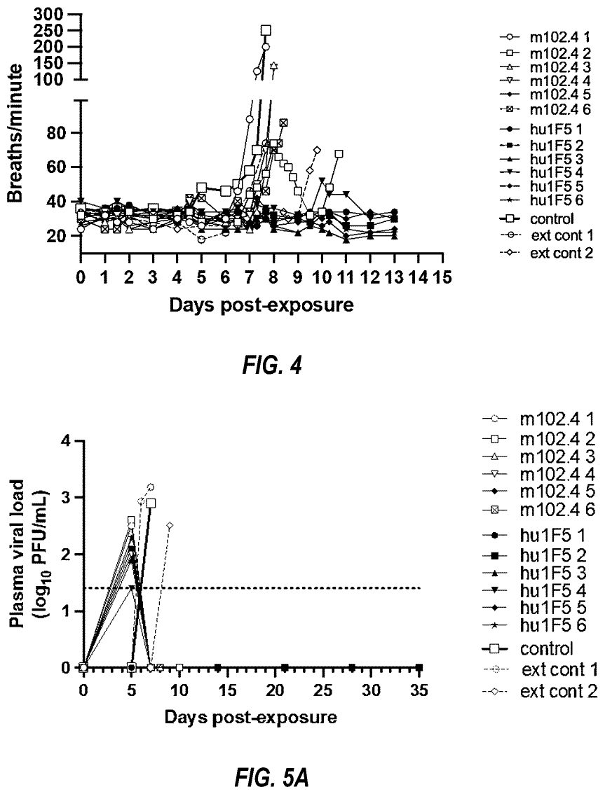

Clinically, control and treated animals that succumbed to disease all displayed respiratory symptoms (labored breathing/dyspnea), and reduced activity and responsiveness (FIG. 3). These animals exhibited increased respiration rates for approximately 24 hours before meeting euthanasia criteria (FIG. 4). The m102.4-treated animals that succumbed also displayed reduced appetite, an observation not noted in the contemporaneous and external controls (FIG. 3). The m102.4-treated animal that survived (m102.4 #5) had reduced appetite but displayed no other clinical symptoms of illness. In contrast, the animals treated with hu1F5 did not display evidence of clinical disease (FIG. 3) or changes in respiration (FIG. 4) except NHP hu1F5 #4 which had a slightly elevated respiration rate for approximately one day starting 10 dpe.

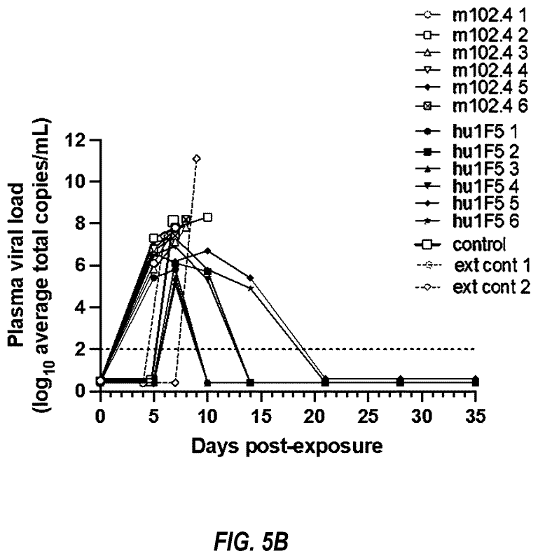

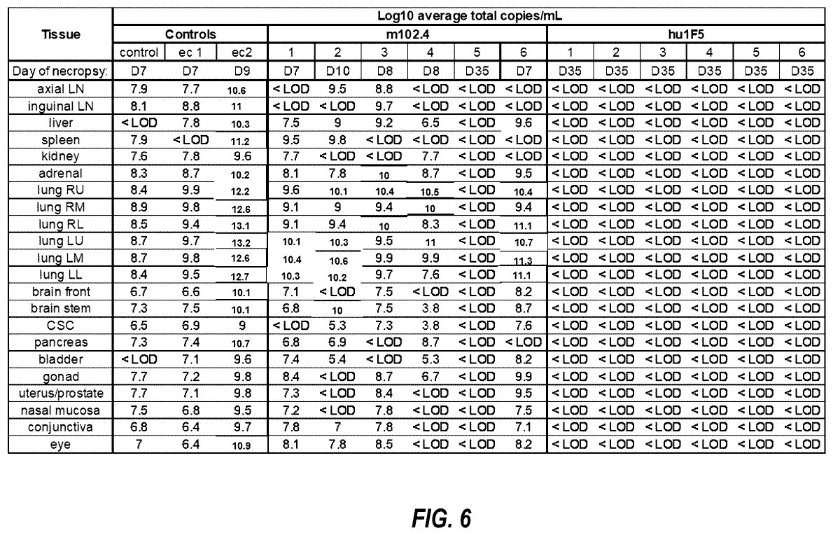

All animals that succumbed to disease had viral load detectable in plasma by plaque assay as well as by qRT-PCR (FIG. 5A-B). Prior to mAb dosing, 8 of 12 treated animals were viremic by plaque assay (4 of 6 in both the m102.4 and hu1F5 treatment groups; FIG. 5A). While the contemporaneous control animal had a high viral titer by plaque assay 7 dpe, the day it succumbed to disease, none of the AGMs treated with either of the neutralizing mAbs had detectable virus by plaque assay on 7 dpe (2 days after dosing), including the four m102.4 treated non-surviving animals. These results suggest the mAbs neutralized any free virus in the plaque assay. By qRT-PCR most of the animals had detectable viral RNA on day 5 prior to treatment: 5 of 6 treated with m102.4 and 4 of 6 treated with hu1F5 (FIG. 5B). All animals had high levels of viral RNA with peak loads detected between 7 and 10 dpe. Animals that survived cleared detectable viral RNA between 10 and 21 dpe.

To assess the systemic viral burden in the animals, qRT-PCR was performed on tissues. Testing of the control animal and external controls (FIG. 6) revealed disseminated infection with high titers present (up to 1013 copies/mL) in all tissues tested except for liver and bladder in the contemporaneous control and the spleen in external control 1. Animals treated with m102.4 that succumbed to disease also had disseminated infection, but with more tissues with undetectable levels of viral RNA than the control animals. NiV RNA was not detected in any tissue of animals that survived until the end of the study.

### Example 4—Efficacy of a Reduced Dose of Hu1F5

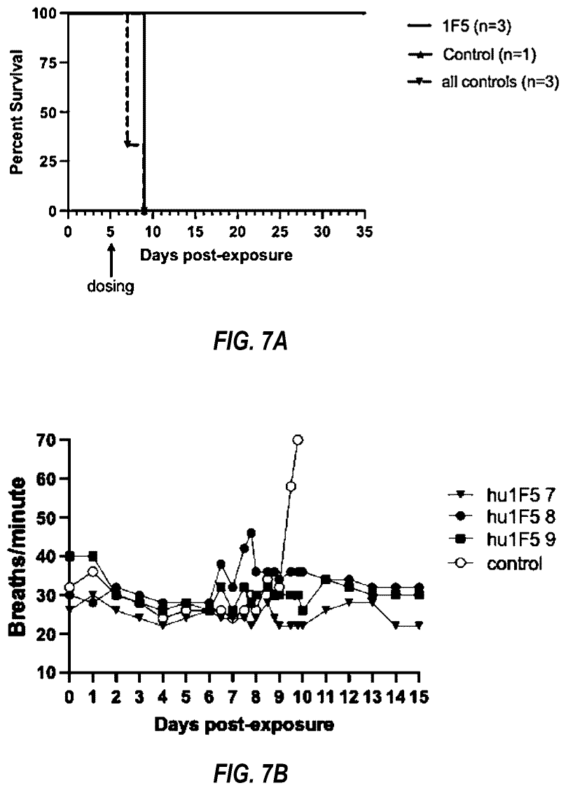

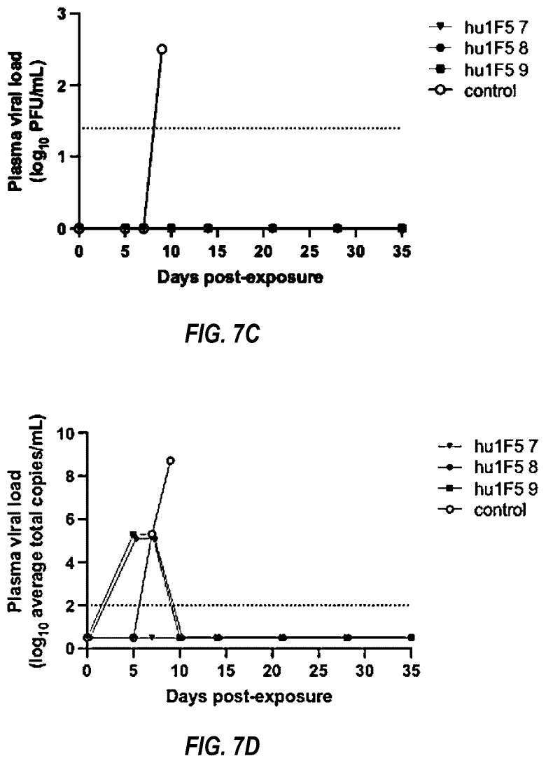

Based on the protection observed in the initial study, a small pilot dose-down experiment was performed in AGMs using an identical challenge protocol as discussed in Example 3 above to determine the effect of reducing the treatment dose to 10 mg/kg. As FIG. 7A illustrates, the three AGMs treated with hu1F5 survived while the contemporaneous control animal (external control 2 in FIG. 3-6) succumbed on day 9 (P=0.08 comparing hu1F5-treated with contemporaneous control by log-rank test; P=0.02 comparing hu1F5-treated with all 3 control animals challenged with the same viral stock using the same protocol). One AGM (labeled hu1F5 8 in FIG. 7B) treated with hu1F5 displayed a mild and transient increase in respiration on days 6, 8 and 9 (FIG. 7B). None of the hu1F5-treated animals displayed any clinical signs of disease. Infectious NiV was not detected at any time in treated animals by plaque assay (FIG. 7C) while 2 of 3 treated animals were positive 5 and 7 dpe by qRT-PCR (FIG. 7D). Finally, NiV RNA was not detected by qRT-PCR in any tested tissue at necropsy (the same tissue panel as in FIG. 6) in hu1F5-treated animals.

### Example 5—Extending Serum Half-Life of Hu1F5

To maximize hu1F5's potential clinical utility as a prophylactic, post-exposure prophylactic, and therapeutic mAb, two mutations were introduced into its Fc region to extend its serum half-life. The mutations are M430L and N436A, and the modified antibody was designated MBP1F5 (HC sequence is SEQ ID NO: 16; LC sequence is SEQ ID NO: 17).

MBP1F5 was tested in an NHP pharmacokinetic (PK) study. The study design was as shown in the Table 1 below. MBP1F5 was administered as a single intramuscular (IM) injection or intravenous (IV) slow bolus dose to NHPs.

TABLE 1
NHP experimental study design.

| Group | Treatment | Route | Dose Level (mg/kg) | Total No. of Animals |
|---|---|---|---|---|
| 1 | MBP1F5 | IM | 1 | 2 Males/ 2 Females |
| 2 | MBP1F5 | IM | 3 | 2 Males/ 2 Females |
| 3 | MBP1F5 | IM | 10 | 2 Males/ 2 Females |
| 4 | MBP1F5 | IV | 10 | 2 Males/ 2 Females |

In NHPs, MBP1F5 was eliminated with a long terminal half-life of about 29 to 42 days after IM doses, and about 37 days after IV administration. In comparison, MBPF15 had an observed half-life of about 14 to 16 days in rodents.

Intramuscular and intravenous delivery of MBP1F5 is being evaluated in a human Phase 1 clinical trial.

### Example 6—MBP1F5 Neutralizes Live Nipah Virus

Neutralization data against authentic NiV_{B} was generated for MBP1F5. The IC₅₀ was 160 ng/mL, which is similar to neutralization activity demonstrated for 1F5 and hu1F5 in previous studies (Table 2).

TABLE 2

| | NiV_{M} IC₅₀ | NiV_{B} IC₅₀ | HeV IC₅₀ |
|---|---|---|---|
| mAb | (ng/mL) | (ng/mL) | (ng/mL) |
| 1F5 (murine)^{a} | 141.2 | 862.0 | 425.2 |
| hu1F5^{a} | 194.1 | 804.2 | 268.5 |
| MBP1F5^{b} | NA | 160.0 | NA |

^{a}Live virus neutralization data from Dang, et al., (2021). “Broadly neutralizing antibody cocktails targeting Nipah virus and Hendra virus fusion glycoproteins.” Nat Struct Mol Biol 28(5): 426-434.
^{b}Live virus neutralization data.
NA: Not applicable.

### Example 7—MBP1F5 does not Cross React with Human Tissues

A GLP tissue cross reactivity study was conducted to determine the potential cross-reactivity of biotin-labeled MBP1F5 with cryosections of human tissues. Biotinylated human immunoglobulin G subclass 1 (IgG1) was used as a control test article. Nipah virus fusion glycoprotein-His tag UV-resin spot slides were used as positive staining controls for the study, while human hypercalcemia of malignancy peptide UV-resin spot slides were used as negative staining controls.

There was no biotin-MBP1F5 binding in the human tissue panels that were examined. This result was anticipated as MBP1F5 binds to a viral antigen that is not expressed in normal human tissues.

### Example 8—MBP1F5 Pharmacology in Hamster Model of Nipah Virus

In addition to efficacy testing of precursor mAbs, a proof-of-concept PrEP efficacy study of MBP1F5 was conducted in a hamster NiV_{B} disease model. This study assessed the survival of Syrian golden hamsters treated by the intramuscular (IM) or intraperitoneal (IP) route with MBP1F5 given 4 days prior to challenge with NiV_{B} (IP, 1×10⁶ PFU). Treatment with a single dose of MBP1F5 delivered by the IM route was protective against lethal NiV_{B} challenge, with doses as low as 0.5 mg/kg conferring complete protection (survival). A dose of 0.25 mg/kg IM was 80% protective against mortality. A dose of 0.25 mg/kg delivered by the IP route was not found to be significantly efficacious in this study (Table 3).

TABLE 3
Summary of Pharmacology in Hamster NiV Disease Model.

| | Pre- or | | | | Intervention | |
|---|---|---|---|---|---|---|
| | Post- | Challenge | | | DPC and Route | |
| | Exposure | strain, route, | | Dose | of Drug | |
| Study | Study | and titer | mAb | (mg/kg) | Administration | Survival |
| Hamster | Pre- | NiV_{B} IP | MBP1F5 | 5 | −4 (IM) | 90% (9 of |
| | Exposure | 1 × 10⁶ PFU | | | | 10) |
| | | | MBP1F5 | 1.5 | −4 (IM) | 100% (6 of |
| | | | | | | 6) |
| | | | MBP1F5 | 0.5 | −4 (IM) | 100% (6 of |
| | | | | | | 6) |
| | | | MBP1F5 | 0.25 | −4 (IM) | 80% (4 of 5) |
| | | | MBP1F5 | 0.25 | −4 (IP) | 33% (1 of 3) |
| | | | Vehicle | 0 | −4 (IM) | 0% (0 of 6) |
| | | | Control | | | |

DPC: Days post-challenge;
IM: Intramuscular;
IN: Intranasal;
IP: Intraperitoneal;
mAb: Monoclonal antibody:
NA: Not applicable;
PFU: Plaque-forming unit

### Example 9—MBP1F5 Pharmacology in Non-Human Primate (NHP) Model of Nipah Virus

In addition to efficacy testing of precursor mAbs, a proof-of-concept PrEP efficacy study of MBP1F5 was conducted in a grivet monkey (Chlorocebus aethiops) disease model. This study evaluated survival rate following pre-exposure prophylactic administration of MBP1F5 when provided via a single intramuscular injection to grivet monkeys challenged with NiV_{B} via the IN and intratracheal route (IN/IT, 5×10⁵ PFU split evenly between routes). MBP1F5 doses of 1, 3, and 10 mg/kg were administered 3 days prior to challenge. 100% survival was observed for all MBP1F5 treatment groups as compared to 20% survival in control animals (Table 4).

TABLE 4
Summary of Pharmacology in Grivet Monkey NiV Disease Model

| | | | | | Intervention | |
|---|---|---|---|---|---|---|
| | Pre- or | | | | Days Post- | |
| | Post- | | | | Challenge and | |
| | Exposure | Virus Strain, | | | Route of | |
| Study | Study | Route, and Titer | mAb | Dose (mg/kg) | Administration | Survival |
| Grivet | Pre- | NiV_{B} | MBP1F5 | 10 | −3 (IM) | 100% (5 of |
| | Exposure | IN/IT | | | | 5) |
| | | 5 × 10⁵ PFU | MBP1F5 | 3 | −3 (IM) | 100% (5 of |
| | | | | | | 5) |
| | | | MBP1F5 | 1 | −3 (IM) | 100% (5 of |
| | | | | | | 5) |
| | | | Vehicle | 0 | −3 (IM) | 20% (1 of 5) |
| | | | Control | | | |

DPC: Days post-challenge;
IgG1: Immunoglobulin G subclass 1;
IM: Intramuscular;
IN: Intranasal;
IT: Intratracheal;
Intraperitoneal;
IV: Intravenous;
mAb: Monoclonal antibody:
MAD: Mucosal atomization device;
NA: Not applicable;
PFU: Plaque-forming unit

### LIST OF REFERENCES

Below is a list of references relevant to the above Examples.
Alfson, K. J., L. E. Avena, G. Worwa, R. Carrion and A. Griffiths (2017). “Development of a Lethal Intranasal Exposure Model of Ebola Virus in the Cynomolgus Macaque.” Viruses 9(11).
Arunkumar, G., R. Chandni, D. T. Mourya, S. K. Singh, R. Sadanandan, P. Sudan, B. Bhargava, P. Nipah Investigators and G. Health Study (2019). “Outbreak Investigation of Nipah Virus Disease in Kerala, India, 2018.” J Infect Dis 219(12): 1867-1878.
Bornholdt, Z. A., A. S. Herbert, C. E. Mire, S. He, R. W. Cross, A. Z. Wec, D. M. Abelson, J. B. Geisbert, R. M. James, M. N. Rahim, W. Zhu, V. Borisevich, L. Banadyga, B. M. Gunn, K. N. Agans, A. S. Wirchnianski, E. Goodwin, K. Tierney, W. S. Shestowsky, O. Bohorov, N. Bohorova, J. Velasco, E. Ailor, D. Kim, M. H. Pauly, K. J. Whaley, G. Alter, L. M. Walker, K. Chandran, L. Zeitlin, X. Qiu, T. W. Geisbert and J. M. Dye (2019). “A Two-Antibody Pan-Ebolavirus Cocktail Confers Broad Therapeutic Protection in Ferrets and Nonhuman Primates.” Cell Host Microbe 25(1): 49-58 e45.
Corti, D., J. Misasi, S. Mulangu, D. A. Stanley, M. Kanekiyo, S. Wollen, A. Ploquin, N. A. Doria-Rose, R. P. Staupe, M. Bailey, W. Shi, M. Choe, H. Marcus, E. A. Thompson, A. Cagigi, C. Silacci, B. Fernandez-Rodriguez, L. Perez, F. Sallusto, F. Vanzetta, G. Agatic, E. Cameroni, N. Kisalu, I. Gordon, J. E. Ledgerwood, J. R. Mascola, B. S. Graham, J. J. Muyembe-Tamfun, J. C. Trefry, A. Lanzavecchia and N. J. Sullivan (2016). “Protective monotherapy against lethal Ebola virus infection by a potently neutralizing antibody.” Science 351(6279): 1339-1342.
Cross, R. W., K. N. Agans, A. N. Prasad, V. Borisevich, C. Woolsey, D. J. Deer, N. S. Dobias, J. B. Geisbert, K. A. Fenton and T. W. Geisbert (2020). “Intranasal exposure of African green monkeys to SARS-CoV-2 results in acute phase pneumonia with shedding and lung injury still present in the early convalescence phase.” Res Sq.
Dang, H. V., R. W. Cross, V. Borisevich, Z. A. Bornholdt, B. R. West, Y. P. Chan, C. E. Mire, S. C. Da Silva, A. S. Dimitrov, L. Yan, M. Amaya, C. K. Navaratnarajah, L. Zeitlin, T. W. Geisbert, C. C. Broder and D. Veesler (2021). “Broadly neutralizing antibody cocktails targeting Nipah virus and Hendra virus fusion glycoproteins.” Nat Struct Mol Biol 28(5): 426-434.
Dong, J., R. W. Cross, M. P. Doyle, N. Kose, J. J. Mousa, E. J. Annand, V. Borisevich, K. N. Agans, R. Sutton, R. Nargi, M. Majedi, K. A. Fenton, W. Reichard, R. G. Bombardi, T. W. Geisbert and J. E. Crowe, Jr. (2020). “Potent Henipavirus Neutralization by Antibodies Recognizing Diverse Sites on Hendra and Nipah Virus Receptor Binding Protein.” Cell 183(6): 1536-1550 e1517.
Geisbert, J. B., V. Borisevich, A. N. Prasad, K. N. Agans, S. L. Foster, D. J. Deer, R. W. Cross, C. E. Mire, T. W. Geisbert and K. A. Fenton (2020). “An Intranasal Exposure Model of Lethal Nipah Virus Infection in African Green Monkeys.” J Infect Dis 221 (Suppl 4): S414-S418.
Geisbert, T. W., K. M. Daddario-DiCaprio, A. C. Hickey, M. A. Smith, Y. P. Chan, L. F. Wang, J. J. Mattapallil, J. B. Geisbert, K. N. Bossart and C. C. Broder (2010). “Development of an acute and highly pathogenic nonhuman primate model of Nipah virus infection.” PLOS One 5(5): e10690.
Geisbert, T. W., H. Feldmann and C. C. Broder (2012). “Animal challenge models of henipavirus infection and pathogenesis.” Curr Top Microbiol Immunol 359:153-177.
Geisbert, T. W., C. E. Mire, J. B. Geisbert, Y. P. Chan, K. N. Agans, F. Feldmann, K. A. Fenton, Z. Zhu, D. S. Dimitrov, D. P. Scott, K. N. Bossart, H. Feldmann and C. C. Broder (2014). “Therapeutic treatment of Nipah virus infection in nonhuman primates with a neutralizing human monoclonal antibody.” Sci Transl Med 6(242): 242ra282.
Gralton, J., E. Tovey, M. L. McLaws and W. D. Rawlinson (2011). “The role of particle size in aerosolised pathogen transmission: a review.” J Infect 62(1): 1-13.
Halpin, K., A. D. Hyatt, R. Fogarty, D. Middleton, J. Bingham, J. H. Epstein, S. A. Rahman, T. Hughes, C. Smith, H. E. Field, P. Daszak and G. Henipavirus Ecology Research (2011). “Pteropid bats are confirmed as the reservoir hosts of henipaviruses: a comprehensive experimental study of virus transmission.” Am J Trop Med Hyg 85(5): 946-951.
Hammoud, D. A., M. R. Lentz, A. Lara, J. K. Bohannon, I. Feuerstein, L. Huzella, P. B. Jahrling, M. Lackemeyer, J. Laux, O. Rojas, P. Sayre, J. Solomon, Y. Cong, V. Munster and M. R. Holbrook (2018). “Aerosol exposure to intermediate size Nipah virus particles induces neurological disease in African green monkeys.” PLOS Negl Trop Dis 12(11): e0006978.
Hayman, D. T., R. Suu-Ire, A. C. Breed, J. A. McEachern, L. Wang, J. L. Wood and A. A. Cunningham (2008). “Evidence of henipavirus infection in West African fruit bats.” PLOS One 3(7): e2739.
Hayman, D. T., L. F. Wang, J. Barr, K. S. Baker, R. Suu-Ire, C. C. Broder, A. A. Cunningham and J. L. Wood (2011). “Antibodies to henipavirus or henipa-like viruses in domestic pigs in Ghana, West Africa.” PLOS One 6(9): e25256.
Luby, S. P. (2013). “The pandemic potential of Nipah virus.” Antiviral Res 100(1): 38-43.
Mire, C. E., B. A. Satterfield, J. B. Geisbert, K. N. Agans, V. Borisevich, L. Yan, Y. P. Chan, R. W. Cross, K. A. Fenton, C. C. Broder and T. W. Geisbert (2016). “Pathogenic Differences between Nipah Virus Bangladesh and Malaysia Strains in Primates: Implications for Antibody Therapy.” Sci Rep 6:30916.
Mire, C. E., B. A. Satterfield and T. W. Geisbert (2023). “Nonhuman Primate Models for Nipah and Hendra Virus Countermeasure Evaluation.” Methods Mol Biol 2682:159-173.
Mustafa, S. S., R. A. Stern, P. C. Patel and D. K. Chu (2023). “COVID-19 Treatments: Then and Now.” J Allergy Clin Immunol Pract.
Playford, E. G., T. Munro, S. M. Mahler, S. Elliott, M. Gerometta, K. L. Hoger, M. L. Jones, P. Griffin, K. D. Lynch, H. Carroll, D. El Saadi, M. E. Gilmour, B. Hughes, K. Hughes, E. Huang, C. de Bakker, R. Klein, M. G. Scher, I. L. Smith, L. F. Wang, S. B. Lambert, D. S. Dimitrov, P. P. Gray and C. C. Broder (2020). “Safety, tolerability, pharmacokinetics, and immunogenicity of a human monoclonal antibody targeting the G glycoprotein of henipaviruses in healthy adults: a first-in-human, randomised, controlled, phase 1 study.” Lancet Infect Dis.
Prasad, A. N., C. Woolsey, J. B. Geisbert, K. N. Agans, V. Borisevich, D. J. Deer, C. E. Mire, R. W. Cross, K. A. Fenton, C. C. Broder and T. W. Geisbert (2020). “Resistance of Cynomolgus Monkeys to Nipah and Hendra Virus Disease Is Associated With Cell-Mediated and Humoral Immunity.” J Infect Dis 221 (Suppl 4): S436-S447.
Singh, R. K., K. Dhama, S. Chakraborty, R. Tiwari, S. Natesan, R. Khandia, A. Munjal, K. S. Vora, S. K. Latheef, K. Karthik, Y. Singh Malik, R. Singh, W. Chaicumpa and D. T. Mourya (2019). “Nipah virus: epidemiology, pathology, immunobiology and advances in diagnosis, vaccine designing and control strategies—a comprehensive review.” Vet Q 39(1): 26-55.
Soman Pillai, V., G. Krishna and M. Valiya Vecttil (2020). “Nipah Virus: Past Outbreaks and Future Containment.” Viruses 12(4).
Van der Gucht, W., K. Stobbelaar, M. Govaerts, T. Mangodt, C. Barbezange, A. Leemans, B. De Winter, S. Van Gucht, G. Caljon, L. Maes, J. De Dooy, P. Jorens, A. Smet, P. Cos, S. Verhulst and P. L. Delputte (2019). “Isolation and Characterization of Clinical RSV Isolates in Belgium during the Winters of 2016-2018.” Viruses 11(11).
Zhu, Q., J. S. Mclellan, N. L. Kallewaard, N. D. Ulbrandt, S. Palaszynski, J. Zhang, B. Moldt, A. Khan, C. Svabek, J. M. McAuliffe, D. Wrapp, N. K. Patel, K. E. Cook, B. W. M. Richter, P. C. Ryan, A. Q. Yuan and J. A. Suzich (2017). “A highly potent extended half-life antibody as a potential RSV vaccine surrogate for all infants.” Sci Transl Med 9(388).
Zhu, Z., K. N. Bossart, K. A. Bishop, G. Crameri, A. S. Dimitrov, J. A. McEachern, Y. Feng, D. Middleton, L. F. Wang, C. C. Broder and D. S. Dimitrov (2008). “Exceptionally potent cross-reactive neutralization of Nipah and Hendra viruses by a human monoclonal antibody.” J Infect Dis 197(6): 846-853.

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

### INFORMAL SEQUENCE LISTING

Sequences as identified in this disclosure are listed below. Unless noted otherwise, amino acid sequences are shown in the N-terminus to C-terminus direction and nucleic acid sequences are in the 5′ to 3′ direction.

| SEQ | | |
|---|---|---|
| ID NO | Description | Sequence |
| 1 | Heavy chain CDR1 | SGYSITSDYYW |
| | (HCDR1) of a humanized | |
| | Henipavirus antibody; | |
| | HCDR1 of hu1F5 | |
| 2 | Heavy chain CDR2 | VTYDGSN |
| | (HCDR2) of a humanized | |
| | Henipavirus antibody; | |
| | HCDR2 of hu1F5 | |
| 3 | Heavy chain CDR3 | RFGSSYWAMDYW |
| | (HCDR3) of a humanized | |
| | Henipavirus antibody; | |
| | HCDR3 of hu1F5 | |
| 4 | Light chain CDR1 (LCDR1) | RASSSVSYMH |
| | of a humanized Henipavirus | |
| | antibody; LCDR1 of hu1F5 | |
| 5 | Light chain CDR2 (LCDR2) | STSNLAS |
| | of a humanized Henipavirus | |
| | antibody; LCDR2 of hu1F5 | |
| 6 | Light chain CDR3 (LCDR3) | HQWYSYPWT |
| | of a humanized Henipavirus | |
| | antibody; LCDR3 of hu1F5 | |
| 7 | Heavy chain variable region | DVQLQESGPGLVKPSDTLSLTCAVSGYSITSDYYWNWIRQPP |
| | (V_{H}) of a humanized | GKGLEWMGYVTYDGSNNYNPSLKSRITISRDSSKNQFSLKLS |
| | Henipavirus antibody; V_{H} of | SVTALDTAVYYCARFGSSYWAMDYWGQGTLVTVSS |
| | hu1F5 | |
| 8 | Light chain variable region | QIQLTQSPSSLSASVGDRVTITCRASSSVSYMHWYQQKPGKA |
| | (V_{L}) of a humanized | PKLLIYSTSNLASGVPSRFSGSGSGTDYTLTISSLQPEDFATYY |
| | Henipavirus antibody; Final | CHQWYSYPWTFGGGTKVEIK |
| | V_{L} of hu1F5 (or h1F5.2) | |
| 9 | Heavy chain variable region | DVQLQESGPGLVKPSQSLSLTCSVTGYSITSDYYWNWIRQFP |
| | (V_{H}) of a murine Henipavirus | GNKLEWMGYVTYDGSNNYNPSLKNRISITRDSSKNQFFLKLN |
| | antibody | SVISEDTATYYCARFGSSYWAMDYWGQGTSLTVSS |
| 10 | Light chain variable region | QIVLTQSPAIMSASLGEAITLTCSASSSVSYMHWYQQKSGTSP |
| | (V_{L}) of a murine Henipavirus | KLLIYSTSNLASGVPSRFSGSGSGTFYSLTISSVEAEDAADYY |
| | antibody | CHQWYSYPWTFGGGTKLEIK |
| 11 | Heavy chain variable region | DVQLVESGPGLVKPGGSLRLTCAVTGYSITSDYYWNWIRQA |
| | (V_{H}) of an exemplary | PGKGLEWMGYVTYDGSNNYNPSLKNRITISRDSSKNTFYLQL |
| | humanized Henipavirus | NSVRAEDTAVYYCARFGSSYWAMDYWGQGTLVTVSS |
| | antibody (initial humanized | |
| | sequence) | |
| 12 | Light chain variable region | QIVLTQSPSSLSASLGERVTITCSASSSVSYMHWYQQKPGQAP |
| | (V_{L}) of an exemplary | KLLIYSTSNLASGVPSRFSGSGSGTDYSLTISSLEAEDFADYYC |
| | humanized Henipavirus | HQWYSYPWTFGGGTKVEIK |
| | antibody (initial humanized | |
| | sequence) | |
| 13 | Heavy chain variable region | DVQLVESGPGLVKPSQSLRLTCAVTGYSITSDYYWNWIRQFP |
| | (V_{H}) of an exemplary | GNKLEWMGYVTYDGSNNYNPSLKNRITISRDSSKNQFFLQLN |
| | humanized Henipavirus | SVRAEDTAVYYCARFGSSYWAMDYWGQGTLLTVSS |
| | antibody (with conservative | |
| | amino acid substitutions) | |
| 14 | Light chain variable region | QIVLTQSPSSMSASLGERITLTCSASSSVSYMHWYQQKPGQA |
| | (V_{L}) of an exemplary | PKLLIYSTSNLASGVPSRFSGSGSGTDYSLTISSVEAEDFADYY |
| | humanized Henipavirus | CHQWYSYPWTFGGGTKLEIK |
| | antibody (with conservative | |
| | amino acid substitutions) | |
| 15 | Final hu1F5 (or h1F5.2) mAb | DVQLQESGPGLVKPSDTLSLTCAVSGYSITSDYYWNWIRQPP |
| | V_{H} sequence | GKGLEWMGYVTYDGSNNYNPSLKSRITISRDSSKNQFSLKLS |
| | | SVTALDTAVYYCARFGSSYWAMDYWGQGTLVTVSS |
| 16 | MBP1F5 DS Heavy Chain | MDMRVPAQLLGLLLLWLRGARCDVQLQESGPGLVKPSDT |
| | (HC) Sequence; underlining | LSLTCAVSGYSITSDYYWNWIRQPPGKGLEWMGYVTYDGSN |
| | indicates the variable region, | NYNPSLKSRITISRDSSKNQFSLKLSSVTALDTAVYYCARFGS |
| | bold letters indicate the | SYWAMDYWGQGTLVTVSSASTKGPSVFPLAPSSKSTSGGTA |
| | leader sequence | ALGCLVKDYFPEPVTVSWNSGALTSGVHTFPAVLQSSGLYSL |
| | | SSVVTVPSSSLGTQTYICNVNHKPSNTKVDKKVEPKSCDKTH |
| | | TCPPCPAPELLGGPSVFLFPPKPKDTLMISRTPEVTCVVVDVS |
| | | HEDPEVKFNWYVDGVEVHNAKTKPREEQYNSTYRVVSVLT |
| | | VLHQDWLNGKEYKCKVSNKALPAPIEKTISKAKGQPREPQV |
| | | YTLPPSRDELTKNQVSLTCLVKGFYPSDIAVEWESNGQPENN |
| | | YKTTPPVLDSDGSFFLYSKLTVDKSRWQQGNVFSCSVLHEAL |
| | | HAHYTQKSLSLSPG |
| 17 | MBP1F5 DS Light Chain | MDMRVPAQLLGLLLLWLRGARCQIQLTQSPSSLSASVGDR |
| | (LC) Sequence; underlining | VTITCRASSSVSYMHWYQQKPGKAPKLLIYSTSNLASGVPSR |
| | indicates the variable region, | FSGSGSGTDYTLTISSLQPEDFATYYCHQWYSYPWTFGGGTK |
| | bold letters indicate the | VEIKRTVAAPSVFIFPPSDEQLKSGTASVVCLLNNFYPREAKV |
| | leader sequence | QWKVDNALQSGNSQESVTEQDSKDSTYSLSSTLTLSKADYE |
| | | KHKVYACEVTHQGLSSPVTKSFNRGEC |
| 18 | Sequence for RT-aPCR | CTGCAGGAGGTGTGCTCACGGAGG |
| | probe | |

1. An isolated humanized antibody that binds to Henipavirus F glycoprotein, wherein the isolated humanized antibody comprises:
(i) a heavy chain variable region (V_{H}) with an amino acid substitution mutation at position 25 relative to a sequence of SEQ ID NO: 9, optionally wherein the V_{H} amino acid substitution mutation is T25S relative SEQ ID NO: 9; and/or
(ii) a light chain variable region (V_{L}) with an amino acid substitution mutation at position 24 relative to a sequence of SEQ ID NO: 10, optionally wherein the V_{L} amino acid substitution mutation is S24R relative to SEQ ID NO: 10.

2. The isolated humanized antibody of claim 1, wherein the isolated humanized antibody binds to an epitope located on a pre-fusion form of the Henipavirus F glycoprotein.

3. The isolated humanized antibody of claim 2, wherein the Henipavirus F glycoprotein is a Nipah virus (NiV) F glycoprotein or a Hendra virus (HeV) F glycoprotein.

4. The isolated humanized antibody of claim 1, wherein the isolated antibody can treat Nipah virus (NiV), Bangladesh strain of NiV (NiVB), Malaysia strain of NiV (NiVM), Cedar henipavirus (CedV), Ghanaian bat henipavirus (Kumasi virus; KV), Langya henipavirus (LayV), Mojiang henipavirus (MojV), and Hendra virus (HeV).

5. The isolated humanized antibody of claim 1, wherein the isolated humanized antibody comprises:
(i) a heavy chain complementarity-determining region (HCDR) comprising:
(a) an HCDR1 comprising a sequence of SEQ ID NO: 1 (SGYSITSDYYW);
(b) an HCDR2 comprising a sequence of SEQ ID NO: 2 (VTYDGSN); and
(c) an HCDR3 comprising a sequence of SEQ ID NO: 3 (RFGSSYWAMDYW); and
(ii) a light chain complementarity-determining region (LCDR) comprising:
(a) an LCDR1 comprising a sequence of SEQ ID NO: 4 (RASSSVSYMH);
(b) an LCDR2 comprising a sequence of SEQ ID NO: 5 (STSNLAS); and
(c) an LCDR3 comprising a sequence of SEQ ID NO: 6 (HQWYSYPWT).

6. The isolated humanized antibody of claim 1, wherein the isolated humanized antibody comprises:
(i) an HCDR1 comprising a sequence of SEQ ID NO: 1 (SGYSITSDYYW);
(ii) an HCDR2 comprising a sequence of SEQ ID NO: 2 (VTYDGSN);
(iii) an HCDR3 comprising a sequence of SEQ ID NO: 3 (RFGSSYWAMDYW);
(iv) an LCDR1 comprising a sequence of SEQ ID NO: 4 (RASSSVSYMH);
(v) an LCDR2 comprising a sequence of SEQ ID NO: 5 (STSNLAS); and
(vi) an LCDR3 comprising a sequence of SEQ ID NO: 6 (HQWYSYPWT).

7. The isolated humanized antibody of claim 1, wherein the V_{H} comprises an amino acid sequence having at least 95% identity to the sequence of SEQ ID NO: 7 or 9, and the V_{L} sequence comprises an amino sequence having at 95% identity to the sequence of SEQ ID NO: 8 or 10.

8. The isolated humanized antibody of claim 1, wherein
(i) the V_{H} comprises at least 95% identity to the sequence of SEQ ID NO: 7 or 9, and
(a) an HCDR1 comprising the sequence of SEQ ID NO: 1;
(b) an HCDR2 comprising the sequence of SEQ ID NO: 2; and
(c) an HCDR3 comprising the sequence of SEQ ID NO: 3; and
(ii) the V_{L} comprises at least 95% identity to the sequence of SEQ ID NO: 8 or 10, and
(a) an LCDR1 comprising the sequence of SEQ ID NO: 4;
(b) an LCDR2 comprising the sequence of SEQ ID NO: 5; and
(c) an LCDR3 comprising the sequence of SEQ ID NO: 6.

9. The isolated humanized antibody of claim 1, wherein at least 1 or 2 of the substitutions are conservative substitutions, at least 50% of the substitutions are conservative substitutions, or all of the substitutions are conservative substitutions.

10. The isolated humanized antibody of claim 1, wherein
(i) the V_{H} sequence comprises the sequence of SEQ ID NO: 7 and (ii) the V_{L} sequence comprises the sequence of SEQ ID NO: 8.

11. (canceled)

12. The isolated humanized antibody of claim 1, wherein (i) the HC sequence comprises an amino acid sequence having at least 95% identity to the sequence of SEQ ID NO: 16, and (ii) the LC sequence comprises an amino acid sequence having at least 95% identity to the sequence of SEQ ID NO: 17.

13. (canceled)

14. The isolated humanized antibody of claim 1, wherein the isolated humanized antibody further comprises at least one Fc mutation, wherein the at least one Fc mutation confers increased half-life.

15. The isolated humanized antibody of claim 14, wherein the at least one Fc mutation is selected from the group consisting of M430L and N436A.

16. An isolated or humanized antibody that binds to the same epitope as and/or competes for binding with the isolated antibody of claim 1.

17. (canceled)

18. (canceled)

19. A recombinant nucleic acid encoding the antibody according to claim 1.

20. An expression vector comprising a polynucleotide encoding a V_{H} sequence and/or a V_{L} sequence of the antibody of claim 1.

21. A host cell that comprises an expression vector of claim 20.

22. A method of producing a humanized antibody, the method comprising culturing a host cell of claim 21 under conditions in which one or more polynucleotides encoding a heavy chain and/or a light chain are expressed.

23. A polypeptide comprising
(i) a V_{H} sequence of SEQ ID NO: 7 or having at least 70% amino acid sequence identity to a sequence of SEQ ID NO: 7, and/or a V_{L} sequence of SEQ ID NO: 8 or having at least 70% amino acid sequence identity to a sequence of SEQ ID NO: 8; or
(ii) a HC sequence of SEQ ID NO: 16 or having at least 70% amino acid sequence identity to a sequence of SEQ ID NO: 16, and/or an LC sequence of SEQ ID NO: 17 or having at least 70% amino acid sequence identity to a sequence of SEQ ID NO: 17.

24. (canceled)

25. (canceled)

26. A pharmaceutical composition comprising the humanized antibody of claim 1 and a pharmaceutically acceptable carrier.

27. A pharmaceutical composition comprising two or more different humanized antibodies of claim 1 and a pharmaceutically acceptable carrier.

28. A method of inducing an immune response and/or treating an infectious disease, the method comprising administering an effective amount of a pharmaceutical composition of claim 26 to a subject in need thereof, wherein (i) the administering step occurs after the subject has been exposed to a Henipavirus that causes the infectious disease and before onset of symptoms of the infectious disease in the subject or (ii) before the subject is exposed to a Henipavirus that causes the infectious disease.

29. (canceled)

30. (canceled)

31. The method of claim 28, wherein the Henipavirus is NiV, NiVB, NiVM, CedV, KV, LayV, MojV, and/or HeV.


SEQUENCE LISTING (18 sequences)

SEQ ID NO: 1 - protein, 11 aa, synthetic construct
SGYSITSDYYW

SEQ ID NO: 2 - protein, 7 aa, synthetic construct
VTYDGSN

SEQ ID NO: 3 - protein, 12 aa, synthetic construct
RFGSSYWAMDYW

SEQ ID NO: 4 - protein, 10 aa, synthetic construct
RASSSVSYMH

SEQ ID NO: 5 - protein, 7 aa, synthetic construct
STSNLAS

SEQ ID NO: 6 - protein, 9 aa, synthetic construct
HQWYSYPWT

SEQ ID NO: 7 - protein, 119 aa, synthetic construct
DVQLQESGPGLVKPSDTLSLTCAVSGYSITSDYYWNWIRQPPGKGLEWMGYVTYDGSNNYNPSLKSRITISRDSSKNQFSLKLSSVTALDTAVYYCARFGSSYWAMDYWGQGTLVTVSS

SEQ ID NO: 8 - protein, 106 aa, synthetic construct
QIQLTQSPSSLSASVGDRVTITCRASSSVSYMHWYQQKPGKAPKLLIYSTSNLASGVPSRFSGSGSGTDYTLTISSLQPEDFATYYCHQWYSYPWTFGGGTKVEIK

SEQ ID NO: 9 - protein, 119 aa, synthetic construct
DVQLQESGPGLVKPSQSLSLTCSVTGYSITSDYYWNWIRQFPGNKLEWMGYVTYDGSNNYNPSLKNRISITRDSSKNQFFLKLNSVISEDTATYYCARFGSSYWAMDYWGQGTSLTVSS

SEQ ID NO: 10 - protein, 106 aa, synthetic construct
QIVLTQSPAIMSASLGEAITLTCSASSSVSYMHWYQQKSGTSPKLLIYSTSNLASGVPSRFSGSGSGTFYSLTISSVEAEDAADYYCHQWYSYPWTFGGGTKLEIK

SEQ ID NO: 11 - protein, 119 aa, synthetic construct
DVQLVESGPGLVKPGGSLRLTCAVTGYSITSDYYWNWIRQAPGKGLEWMGYVTYDGSNNYNPSLKNRITISRDSSKNTFYLQLNSVRAEDTAVYYCARFGSSYWAMDYWGQGTLVTVSS

SEQ ID NO: 12 - protein, 106 aa, synthetic construct
QIVLTQSPSSLSASLGERVTITCSASSSVSYMHWYQQKPGQAPKLLIYSTSNLASGVPSRFSGSGSGTDYSLTISSLEAEDFADYYCHQWYSYPWTFGGGTKVEIK

SEQ ID NO: 13 - protein, 119 aa, synthetic construct
DVQLVESGPGLVKPSQSLRLTCAVTGYSITSDYYWNWIRQFPGNKLEWMGYVTYDGSNNYNPSLKNRITISRDSSKNQFFLQLNSVRAEDTAVYYCARFGSSYWAMDYWGQGTLLTVSS

SEQ ID NO: 14 - protein, 106 aa, synthetic construct
QIVLTQSPSSMSASLGERITLTCSASSSVSYMHWYQQKPGQAPKLLIYSTSNLASGVPSRFSGSGSGTDYSLTISSVEAEDFADYYCHQWYSYPWTFGGGTKLEIK

SEQ ID NO: 15 - protein, 119 aa, synthetic construct; identical to SEQ ID NO: 7

SEQ ID NO: 16 - protein, 470 aa, synthetic construct
MDMRVPAQLLGLLLLWLRGARCDVQLQESGPGLVKPSDTLSLTCAVSGYSITSDYYWNWIRQPPGKGLEWMGYVTYDGSNNYNPSLKSRITISRDSSKNQFSLKLSSVTALDTAVYYCARFGSSYWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 17 - protein, 235 aa, synthetic construct
MDMRVPAQLLGLLLLWLRGARCQIQLTQSPSSLSASVGDRVTITCRASSSVSYMHWYQQKPGKAPKLLIYSTSNLASGVPSRFSGSGSGTDYTLTISSLQPEDFATYYCHQWYSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 24 bp, synthetic construct
  source 1..24: mol_type = other DNA
ctgcaggaggtgtgctcacggagg